(19) United States
(12) Patent Application Publication
(10) Pub. No.: US 2006/0188498 A1
(43) Pub. Date: Aug. 24, 2006

(54) Methods of using death receptor agonists and EGFR inhibitors

(75) Inventor: Avi J. Ashkenazi, San Mateo, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 11/061,258

(22) Filed: Feb. 18, 2005

(51) Int. Cl. A61K 39/395; A61K 31/517; A61K 38/17

(52) U.S. Cl. 424/143.1; 514/12; 514/266.4

(57) ABSTRACT

Methods for using death receptor ligands, such as Apo-2 ligand/TRAIL polypeptides or death receptor antibodies, and EGFR inhibitors to treat pathological conditions such as cancer are provided. Embodiments of the invention include methods of using Apo2L/TRAIL or death receptor antibodies such as DR5 antibodies and DR4 antibodies in combination with EGFR inhibitors, such as Tarceva™.

### FIELD OF THE INVENTION

The present invention relates to methods of using death receptor agonist molecules and EGFR inhibitors. More particularly, the invention relates to methods of using molecules such as Apo-2 ligand/TRAIL or DR4 or DR5 agonist antibodies and EGFR inibitors to treat various pathological disorders, such as cancer.

### BACKGROUND OF THE INVENTION

Control of cell numbers in mammals is believed to be determined, in part, by a balance between cell proliferation and cell death. One form of cell death, sometimes referred to as necrotic cell death, is typically characterized as a pathologic form of cell death resulting from some trauma or cellular injury. In contrast, there is another, “physiologic” form of cell death which usually proceeds in an orderly or controlled manner. This orderly or controlled form of cell death is often referred to as “apoptosis” [see, e.g., Barr et al., Bio/Technology, 12:487-493 (1994); Steller et al., Science, 267:1445-1449 (1995)]. Apoptotic cell death naturally occurs in many physiological processes, including embryonic development and clonal selection in the immune system [Itoh et al., Cell, 66:233-243 (1991)].

Various molecules, such as tumor necrosis factor-alpha (“TNF-alpha”), tumor necrosis factor-beta (“TNF-beta” or “lymphotoxin-alpha”), lymphotoxin-beta (“LT-beta”), CD30 ligand, CD27 ligand, CD40 ligand, OX-40 ligand, 4-1BB ligand, Apo-1 ligand (also referred to as Fas ligand or CD95 ligand), Apo-2 ligand (also referred to as Apo2L or TRAIL), Apo-3 ligand (also referred to as TWEAK), APRIL, OPG ligand (also referred to as RANK ligand, ODF, or TRANCE), and TALL-1 (also referred to as BlyS, BAFF or THANK) have been identified as members of the tumor necrosis factor (“TNF”) family of cytokines [See, e.g., Gruss and Dower, Blood, 85:3378-3404 (1995); Schmid et al., Proc. Natl. Acad. Sci., 83:1881 (1986); Dealtry et al., Eur. J. Immunol., 17:689 (1987); Pitti et al., J. Biol. Chem., 271:12687-12690 (1996); Wiley et al., Immunity, 3:673-682 (1995); Browning et al., Cell, 72:847-856 (1993); Armitage et al. Nature, 357:80-82 (1992), WO 97/01633 published Jan. 16, 1997; WO 97/25428 published Jul. 17, 1997; Marsters et al., Curr. Biol., 8:525-528 (1998); Chicheportiche et al., Biol. Chem., 272:32401-32410 (1997); Hahne et al., J. Exp. Med., 188:1185-1190 (1998); WO98/28426 published Jul. 2, 1998; WO98/46751 published Oct. 22, 1998; WO/98/18921 published May 7, 1998; Moore et al., Science, 285:260-263 (1999); Shu et al., J. Leukocyte Biol., 65:680 (1999); Schneider et al., J. Exp. Med., 189:1747-1756 (1999); Mukhopadhyay et al., J. Biol. Chem., 274:15978-15981 (1999)]. Among these molecules, TNF-alpha, TNF-beta, CD30 ligand, 4-1BB ligand, Apo-1 ligand, Apo-2 ligand (Apo2L/TRAIL) and Apo-3 ligand (TWEAK) have been reported to be involved in apoptotic cell death.

Apo2L/TRAIL was identified several years ago as a member of the TNF family of cytokines. (see, e.g., Wiley et al., Immunity, 3:673-682 (1995); Pitti et al., J. Biol. Chem., 271:12697-12690 (1996); U.S. Pat. No. 6,284,236 issued Sep. 4, 2001] The full-length native sequence human Apo2L/TRAIL polypeptide is a 281 amino acid long, Type II transmembrane protein. Some cells can produce a natural soluble form of the polypeptide, through enzymatic cleavage of the polypeptide's extracellular region (Mariani et al., J. Cell. Biol., 137:221-229 (1997)]. Crystallographic studies of soluble forms of Apo2L/TRAIL reveal a homotrimeric structure similar to the structures of TNF and other related proteins [Hymowitz et al., Molec. Cell, 4:563-571 (1999); Hymowitz et al., Biochemistry, 39:633-644 (2000)]. Apo2L/TRAIL, unlike other TNF family members however, was found to have a unique structural feature in that three cysteine residues (at position 230 of each subunit in the homotrimer) together coordinate a zinc atom, and that the zinc binding is important for trimer stability and biological activity. [Hymowitz et al., supra; Bodmer et al., J. Biol. Chem., 275:20632-20637 (2000))

It has been reported in the literature that Apo2L/TRAIL may play a role in immune system modulation, including autoimmune diseases such as rheumatoid arthritis (see, e.g., Thomas et al., J. Immunol., 161:2195-2200 (1998); Johnsen et al., Cytokine, 11:664-672 (1999); Griffith et al., J. Exp. Med., 189:1343-1353 (1999); Song et al., J. Exp. Med., 191:1095-1103 (2000)].

Soluble forms of Apo2L/TRAIL have also been reported to induce apoptosis in a variety of cancer cells in vitro, including colon, lung, breast, prostate, bladder, kidney, ovarian and brain tumors, as well as melanoma, leukemia, and multiple myeloma [see, e.g., Wiley et al., supra; Pitti et al., supra; Rieger et al., FEBS Letters, 427:124-128 (1998); Ashkenazi et al., J. Clin. Invest., 104:155-162 (1999); Walczak et al., Nature Med., 5:157-163 (1999); Keane et al., Cancer Research, 59:734-741 (1999); Mizutani et al., Clin. Cancer Res., 5:2605-2612 (1999); Gazitt, Leukemia, 13:1817-1824 (1999); Yu et al., Cancer Res., 60:2384-2389 (2000); Chinnaiyan et al., Proc. Natl. Acad. Sci., 97:1754-1759 (2000)). In vivo studies in murine tumor models further suggest that Apo2L/TRAIL, alone or in combination with chemotherapy or radiation therapy, can exert substantial anti-tumor effects [see, e.g., Ashkenazi et al., supra; Walzcak et al., supra; Gliniak et al., Cancer Res., 59:6153-6158 (1999); Chinnaiyan et al., supra; Roth et al., Biochem. Biophys. Res. Comm., 265:1999 (1999)]. In contrast to many types of cancer cells, most normal human cell types appear to be resistant to apoptosis induction by certain recombinant forms of Apo2L/TRAIL [Ashkenazi et al., supra; Walzcak et al., supral. Jo et al. has reported that a polyhistidine-tagged soluble form of Apo2L/TRAIL induced apoptosis in vitro in normal isolated human, but not non-human, hepatocytes [Jo et al., Nature Med., 6:564-567 (2000); see also, Nagata, Nature Med., 6:502-503 (2000)]. It is believed that certain recombinant Apo2L/TRAIL preparations may vary in terms of biochemical properties and biological activities on diseased versus normal cells, depending, for example, on the presence or absence of a tag molecule, zinc content, and % trimer content [See, Lawrence et al., Nature Med., Letter to the Editor, 7:383-385 (2001); Qin et al., Nature Med., Letter to the Editor, 7:385-386 (2001)].

The TNF family ligands identified to date, with the exception of lymphotoxin-α, are type II transmembrane proteins, whose C-terminus is extracellular. In contrast, most receptors in the TNF receptor (TNFR) family identified to date are type I transmembrane proteins. In both the TNF ligand and receptor families, however, homology identified between family members has been found mainly in the extracellular domain (“ECD”). Several of the TNF family cytokines, including TNF-α, Apo-1 ligand and CD40 ligand, are cleaved proteolytically at the cell surface; the resulting protein in each case typically forms a homotrimeric molecule that functions as a soluble cytokine. TNF receptor family proteins are also usually cleaved proteolytically to release soluble receptor ECDs that can function as inhibitors of the cognate cytokines.

Pan et al. have disclosed a TNF receptor family member referred to as “DR4” [Pan et al., Science, 276:111-113 (1997)]. The DR4 was reported to contain a cytoplasmic death domain capable of engaging the cell suicide apparatus. Pan et al. disclose that DR4 is believed to be a receptor for the ligand known as Apo-2 ligand or TRAIL.

In Sheridan et al., Science, 277:818-821 (1997) and Pan et al., Science, 277:815-818 (1997), another molecule believed to be a receptor for Apo2L/TRAIL is described [see also, WO98/51793 published Nov. 19, 1998; WO98/41629 published Sep. 24, 1998]. That molecule is referred to as DR5 (it has also been alternatively referred to as Apo-2; TRAIL-R, TR6, Tango-63, hAPO8, TRICK2 or KILLER [Screaton et al., Curr. Biol., 7:693-696 (1997); Walczak et al., EMBO J., 16:5386-5387 (1997); Wu et al., Nature Genetics, 17:141-143 (1997); WO98/35986 published Aug. 20, 1998; EP870,827 published Oct. 14, 1998; WO98/46643 published Oct. 22, 1998; WO99/02653 published Jan. 21, 1999; WO99/09165 published Feb. 25, 1999; WO99/11791 published Mar. 11, 1999]. Like DR4, DR5 is reported to contain a cytoplasmic death domain and be capable of signaling apoptosis. The crystal structure of the complex formed between Apo-2L/TRAIL and DR5 is described in Hymowitz et al., Molecular Cell, 4:563-571 (1999).

A further group of recently identified TNFR family members are referred to as “decoy receptors,” which are believed to function as inhibitors, rather than transducers of signaling. This group includes DCR1 (also referred to as TRID, LIT or TRAIL-R3) (Pan et al., Science, 276:111-113 (1997); Sheridan et al., Science, 277:818-821 (1997); McFarlane et al., J. Biol. Chem., 272:25417-25420 (1997); Schneider et al., FEBS Letters, 416:329-334 (1997); Degli-Esposti et al., J. Exp. Med., 186:1165-1170 (1997); and Mongkolsapaya et al., J. Immunol., 160:3-6 (1998)] and DCR2 (also called TRUNDD or TRAIL-R4) [Marsters et al., Curr. Biol., 7:1003-1006 (1997); Pan et al., FEBS Letters, 424:41-45 (1998); Degli-Esposti et al., Immunity, 7:813-820 (1997)], both cell surface molecules, as well as OPG (Simonet et al., supra] and DCR3 [Pitti et al., Nature, 396:699-703 (1998)], both of which are secreted, soluble proteins. Apo2L/TRAIL has been reported to bind those receptors referred to as DcR1, DcR2 and OPG.

Apo2L/TRAIL is believed to act through the cell surface “death receptors” DR4 and DR5 to activate caspases, or enzymes that carry out the intracellular cell death program. [See, e.g., Salvesen et al., Cell, 91:443-446 (1997)]. Upon ligand binding, both DR4 and DR5 can trigger apoptosis independently by recruiting and activating the apoptosis initiator, caspase-8, through the death-domain-containing adaptor molecule referred to as FADD/Mort1 [Kischkel et al., Immunity, 12:611-620 (2000); Sprick et al., Immunity, 12:599-609 (2000); Bodmer et al., Nature Cell Biol., 2:241-243 (2000)]. In contrast to DR4 and DR5, the DcR1 and DcR2 receptors do not signal apoptosis.

Certain antibodies which bind to the DR4 and/or DR5 receptors have been reported in the literature. For example, anti-DR4 antibodies directed to the DR4 receptor and having agonistic or apoptotic activity in certain mammalian cells are described in, e.g., WO 99/37684 published Jul. 29, 1999; WO 00/73349 published Jul. 12, 2000; WO 03/066661 published Aug. 14, 2003. See, also, e.g., Griffith et al., J. Immunol., 162:2597-2605 (1999); Chuntharapai et al., J. Immunol., 166:4891-4898 (2001); WO 02/097033 published Dec. 2, 2002; WO 03/042367 published May 22, 2003; WO 03/038043 published May 8, 2003; WO 03/037913 published May 8, 2003. Certain anti-DR5 antibodies have likewise been described, see, e.g., WO 98/51793 published Nov. 8, 1998; Griffith et al., J. Immunol., 162:2597-2605 (1999); Ichikawa et al., Nature Med., 7:954-960 (2001); Hylander et al., “An Antibody to DR5 (TRAIL-Receptor 2) Suppresses the Growth of Patient Derived Gastrointestinal Tumors Grown in SCID mice”, Abstract, 2d International Congress on Monoclonal Antibodies in Cancers, Aug. 29-Sep. 1, 2002, Banff, Alberta, Canada; WO 03/038043 published May 8, 2003; WO 03/037913 published May 8, 2003. In addition, certain antibodies having cross-reactivity to both DR4 and DR5 receptors have been described (see, e.g., US patent 6,252,050 issued Jun. 26, 2001).

For a review of the TNF family of cytokines and their receptors, see Ashkenazi and Dixit, Science, 281:1305-1308 (1998); Ashkenazi and Dixit, Curr. Opin. Cell Biol., 11:255-260 (2000); Golstein, Curr. Biol., 7:750-753 (1997); Gruss and Dower, supra, and Nagata, Cell, 88:355-365 (1997); Locksley et al., Cell, 104:487-501 (2001); Wallach, “TNF Ligand and TNF/NGF Receptor Families”, Cytokine Research, Academic Press, pages 377-411 (2000).

Epidermal Growth Factor Receptor (EGFR) is a member of the type 1 tyrosine kinase family of growth factor receptors, which play critical roles in cellular growth, differentiation, and survival. Activation of these receptors typically occurs via specific ligand binding, resulting in hetero- or homodimerization between receptor family members, with subsequent autophosphorylation of the tyrosine kinase domain. This activation triggers a cascade of intracellular signaling pathways involved in both cellular proliferation (the ras/raf/MAP kinase pathway) and survival (the PI3 kinase/Akt pathway). Members of this family, including EGFR and HER2, have been directly implicated in cellular transformation.

A number of human malignancies are associated with aberrant or overexpression of EGFR and/or overexpression of its specific ligands e.g. transforming growth factor α (Gullick, Br Med Bull 1991, 47:87-98; Modijtahedi and Dean, Int J Oncol 1994, 4:277-96; Salomon et al., Crit Rev Oncol Hematol 1995;19:183-232). EGFR overexpression has been associated with an adverse prognosis in a number of human cancers, including NSCLC. In some instances, overexpression of tumor EGFR has been correlated with both chemoresistance and a poor prognosis (Lei et al., Anticancer Res 1999; 19:221-8; Veale et al., Br J Cancer 1993;68:162-5).

### SUMMARY OF THE INVENTION

The present invention provides methods of enhancing apoptosis in mammalian cells, comprising contacting said cells with an effective amount of a death receptor agonist and an EGFR inhibitor. Optionally, the death receptor agonist is Apo2L/TRAIL polypeptide.

In other embodiments, there are provided methods of treating disorders such as cancer, in a mammal, comprising administering to said mammal an effective amount of death receptor agonist and an EGFR inhibitor.

In further embodiments, there are provided articles of manufacture and kits containing, e.g., Apo2L/TRAIL polypeptide and an EGFR inhibitor useful for the treatment of various pathological disorders.

In more particular embodiments, but without limitations thereto, there are provided the following.exemplary compositions and methods:

1. A method of enhancing apoptosis in one or more mammalian cells, comprising exposing said cells to an effective amount of death receptor agonist and EGFR inhibitor.

2. The method of claim 1, wherein said cells are exposed sequentially to the death receptor agonist and the EGFR inhibitor.

3. The method of claim 1, wherein said cells are exposed to the EGFR inhibitor prior to being exposed to the death receptor agonist.

4. The method of claim 1, wherein said death receptor agonist comprises Apo2L/TRAIL polypeptide.

5. The method of claim 1, wherein said cells are exposed simultaneously to the EGFR inhibitor and the death receptor agonist.

6. The method of claim 1, wherein said death receptor agonist is a DR4 agonist antibody or DR5 agonist antibody.

7. The method of claim 1, wherein said EGFR inhibitor has the general formula I 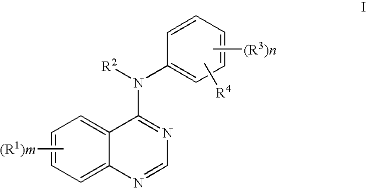
wherein:
X is halo or hydroxy;
m is 1, 2, or 3;
each R¹ is independently selected from the group consisting of hydrogen, halo, hydroxy, hydroxyamino, carboxy, nitro, guanidino, ureido, cyano, trifluoromethyl, and —(C₁ -C₄ alkylene)-W-(phenyl) wherein W is a single bond, O, S or NH;
or each R¹ is independently selected from R⁹ and C₁-C₄ alkyl substituted by cyano, wherein R⁹ is selected from the group consisting of R⁵, —OR⁶, —NR⁶R⁶, —C(O)R⁷, —NHOR⁵, —OC(O)R⁶, cyano, A and —YR⁵; R⁵ is C₁-C₄ alkyl; R⁶ is independently hydrogen or R⁶; R⁷ is R⁵, —OR⁶ or —NR⁶R⁶ ; A is selected from piperidino, morpholino, pyrrolidino, 4-R⁶-piperazin-1-yl, imidazol-1-yl, 4-pyridon-1-yl, —(C₁ -C₄ alkylene) (CO2H), phenoxy, phenyl, phenylsulfanyl, C₂-C₄ alkenyl, and —(C₁-C₄ alkylene)C(O)NR⁶R⁶; and Y is S, SO, or SO₂; wherein the alkyl moieties in R⁵, —OR⁶ and —NR⁶R⁶ are optionally substituted by one to three halo substituents and the alkyl moieties in R⁵, —OR⁶ and —NR⁶R⁶ are optionally substituted by 1 or 2 R⁹ groups, and wherein the alkyl moieties of said optional substituents are optionally substituted by halo or R⁹, with the proviso that two heteroatoms are not attached to the same carbon atom;
or each R¹ is independently selected from —NHSO₂R⁵, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino wherein R¹⁰ is selected from halo, —OR⁶, C₂-C₄ alkanoyloxy, —C(O)R⁷, and —NR⁶R⁶; and wherein said —NHSO₂R⁵, phthalimido-(C₁-C₄-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino R¹ groups are optionally substituted by 1 or 2 substituents independently selected from halo, C₁-C₄ alkyl, cyano, methanesulfonyl and C₁-C₄ alkoxy;
or two R¹ groups are taken together with the carbons to which they are attached to form a 5-8 membered ring that includes 1 or 2 heteroatoms selected from O, S and N;
R² is hydrogen or C₁-C₆ alkyl optionally substituted by 1 to 3 substituents independently selected from halo, C₁-C₄ alkoxy, —NR⁶R⁶, and —SO₂R⁵;
n is 1 or 2 and each R³ is independently selected from hydrogen, halo, hydroxy, C₁-C₆ alkyl, —NR⁶R⁶, and C₁-C₄ alkoxy, wherein the alkyl moieties of said R³ groups are optionally substituted by 1 to 3 substituents independently selected from halo, C₁-C₄ alkoxy, —NR⁶R⁶, and —SO₂R; and,
R⁴ is azido or -(ethynyl)-R¹¹ wherein R¹¹ is hydrogen or C₁-C₆ alkyl optionally substituted by hydroxy, —OR⁶, or —NR⁶R⁶.

8. The method of claim 7, wherein said EGFR inhibitor is N-(3-ethynylphenyl)-6,7-bis(2-methoxyethoxy)-4-quinazolinamine.

9. The method of claim 7, wherein said EGFR inhibitor is Tarceva™.

10. The method of claim 1, wherein said Apo2L/TRAIL is a fragment of the polypeptide of SEQ ID NO:1.

11. The method of claim 10, wherein said Apo2L/TRAIL fragment comprises the extracellular domain of the polypeptide of SEQ ID NO:1.

12. The method of claim 1, wherein said death receptor agonist is an Apo2L/TRAIL polypeptide variant having at least about 90% amino acid sequence identity with the extracellular domain of SEQ ID NO:1.

13. The method of claim 10, wherein said fragment comprises amino acids 114-281 of SEQ ID NO:1.

14. The method of claim 12, wherein said Apo2L/TRAIL variant has at least about 95% amino acid sequence identity with the extracellular domain of SEQ ID NO:1.

15. The method of claim 13, wherein said Apo2L/TRAIL fragment comprising amino acids 114-281 of SEQ ID NO:1 is linked to one or more polyethylene glycol (PEG) molecules.

16. A method of treating a proliferative disorder in a mammal comprising administering to said mammal Apo2L/TRAIL and an EGFR inhibitor.

17. The method of claim 16, wherein said Apo2L/TRAIL and an EGFR inhibitor are administered simultaneously.

18. The method of claim 16, wherein said Apo2L/TRAIL is administered prior to said EGFR inhibitor

19. The method of claim 16, wherein said EGFR inhibitor is administered prior to said Apo2L/TRAIL.

20. The method of claim 1 wherein said proliferative disorder is cancer.

21. The method of claim 20, wherein said cancer is selected from the group consisting of small-cell lung cancer, non-small cell lung cancer, colon cancer, colorectal cancer, and pancreatic cancer.

22. The method of claim 21, wherein said cancer is colon cancer, colorectal cancer, small-cell lung cancer or non-small cell lung cancer.

23. The method of claim 16, wherein said EGFR inhibitor is a compound of the general formula I: 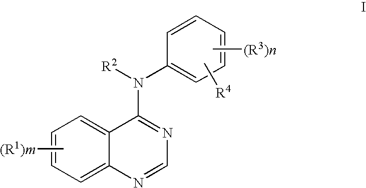
wherein:
X is halo or hydroxy;
m is 1, 2, or 3;
each R¹ is independently selected from the group consisting of hydrogen, halo, hydroxy, hydroxyamino, carboxy, nitro, guanidino, ureido, cyano, trifluoromethyl, and —(C₁ -C₄ alkylene)-W-(phenyl) wherein W is a single bond, O, S or NH;
or each R¹ is independently selected from R⁹ and C₁-C₄ alkyl substituted by cyano, wherein R⁹ is selected from the group consisting of R⁵, —OR⁶, —NR⁶ R⁶, —C(O)R⁷, —NHOR⁵, —OC(O)R⁶, cyano, A and —YR⁵; R⁵ is C₁-C₄ alkyl; R⁶ is independently hydrogen or R⁵; R⁷ is R⁵, —OR⁶ or —NR⁶R⁶ ; A is selected from piperidino, morpholino, pyrrolidino, 4-R⁶-piperazin-1-yl, imidazol-1-yl, 4-pyridon-1-yl, —(C₁-C₄ alkylene) (CO2H), phenoxy, phenyl, phenylsulfanyl, C₂-C₄ alkenyl, and —(C₁-C₄ alkylene)C(O)NR⁶R⁶; and Y is S, SO, or SO₂; wherein the alkyl moieties in R⁵, —OR⁶ and —NR⁶R⁶ are optionally substituted by one to three halo substituents and the alkyl moieties in R⁵, —OR⁶ and —NR⁶R⁶ are optionally substituted by 1 or 2 R⁹ groups, and wherein the alkyl moieties of said optional substituents are optionally substituted by halo or R⁹, with the proviso that two heteroatoms are not attached to the same carbon atom;
or each R¹ is independently selected from —NHSO₂R⁵, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄) -alkanoylamino wherein R¹⁰ is selected from halo, OR⁶, C₂-C₄ alkanoyloxy, —C(O)R⁷, and —NR⁶R⁶; and wherein said —NHSO₂R⁵, phthalimido-(C₁-C₄-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄) -alkanoylamino R¹ groups are optionally substituted by 1 or 2 substituents independently selected from halo, C₁-C₄ alkyl, cyano, methanesulfonyl and C₁-C₄ alkoxy;
or two R¹ groups are taken together with the carbons to which they are attached to form a 5-8 membered ring that includes 1 or 2 heteroatoms selected from O, S and N;
R² is hydrogen or C₁-C₆ alkyl optionally substituted by 1 to 3 substituents independently selected from halo, C₁-C₄ alkoxy, —NR⁶R⁶, and —SO₂R⁵;
n is 1 or 2 and each R³ is independently selected from hydrogen, halo, hydroxy, C₁-C₆ alkyl, —NR⁶R⁶, and C₁-C₄ alkoxy, wherein the alkyl moieties of said R³ groups are optionally substituted by 1 to 3 substituents independently selected from halo, C₁-C₄ alkoxy, —NR⁶R⁶, and —SO₂R; and,
R⁴ is azido or -(ethynyl)-R¹¹ wherein R¹¹ is hydrogen or C₁-C₆ alkyl optionally substituted by hydroxy, —OR⁶, or —NR⁶R⁶.

24. The method of claim 16, wherein said EGFR inhibitor is N-(3-ethynylphenyl)-6,7-bis(2-methoxyethoxy)-4-quinazolinamine.

25. The method of claim 16, wherein said EGFR inhibitor is Tarceva™.

26. A method of treating cancer cells, comprising exposing mammalian cancer cells to a synergistic effective amount of death receptor agonist and EGFR inhibitor.

27. The method of claim 26 wherein said death receptor agonist is an anti-DR5 or anti-DR4 receptor monoclonal antibody.

28. The method of claim 26 wherein said death receptor agonist is Apo-2/TRAIL polypeptide.

29. The method of claim 26 wherein said cancer cells are exposed to said synergistic effective amount of death receptor agonist and EGFR inhibitor in vivo.

30. The method of claim 27 wherein said death receptor antibody is a chimeric antibody or a humanized antibody.

31. The method of claim 27 death receptor antibody is a human antibody.

32. The method of claim 26 wherein said death receptor agonist is an antibody which cross-reacts with more than one Apo-2 ligand receptor.

33. The method of claim 26 wherein said cancer cells are colon cancer cells, colorectal cancer cells, small-cell lung cancer cells or non-small cell lung cancer cells.

34. The method of claim 26 further comprising exposing the cancer cells to one or more growth inhibitory agents.

35. The method of claim 26 further comprising exposing the cells to radiation.

36. The method of claim 27 wherein said DR5 antibody has a DR5 receptor binding affinity of 10⁸ M⁻¹ to 10¹² M⁻¹.

37. The method of claim 26 wherein said death receptor agonist is expressed in a recombinant host cell selected from the group consisting of a CHO cell, yeast cell and E. coli.

38. The method of claim 26 wherein said EGFR inhibitor is Tarceva™.

### BRIEF DESCRIPTION OF THE DRAWINGS

FIG. 1A shows the nucleotide sequence of human Apo-2 ligand cDNA (SEQ ID NO:2) and its derived amino acid sequence (SEQ ID NO:l). The “N” at nucleotide position 447 is used to indicate the nucleotide base may be a “T” or “G”.

FIGS. 2A and 2B show the nucleotide sequence of a cDNA (SEQ ID NO:4) for full length human DR4 and its derived amino acid sequence (SEQ ID NO:3). The respective nucleotide and amino acid sequences for human DR4 are also reported in Pan et al., Science, 276:111 (1997).

FIG. 3A shows the 411 amino acid sequence of human DRS (SEQ ID NO:5) as published in WO 98/51793 on Nov. 19, 1998. A transcriptional splice variant of human DR5 is known in the art. This DR5 splice variant encodes the 440 amino acid sequence of human DR5 (SEQ ID NO:6) shown in FIGS. 3B and 3C as published in WO 98/35986 on Aug. 20, 1998.

FIG. 4 shows respective receptor expression levels (DR4, DR5, EGFR) in H460 cells with or without pre-treatment with Tarceva™ or Taxol®.

FIGS. 5A-5D illustrate effects of Apo2 ligand and Tarceva™ treatments on various cancer cell lines in vitro.

FIG. 6 illustrates effects of Apo2 ligand and Tarceva™ treatments on H460 cancer cells in vivo.

### DETAILED DESCRIPTION OF THE PREFERRED EMBODIMENTS

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

Before the present methods, kits and uses therefor are described, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise.

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

### I. Definitions

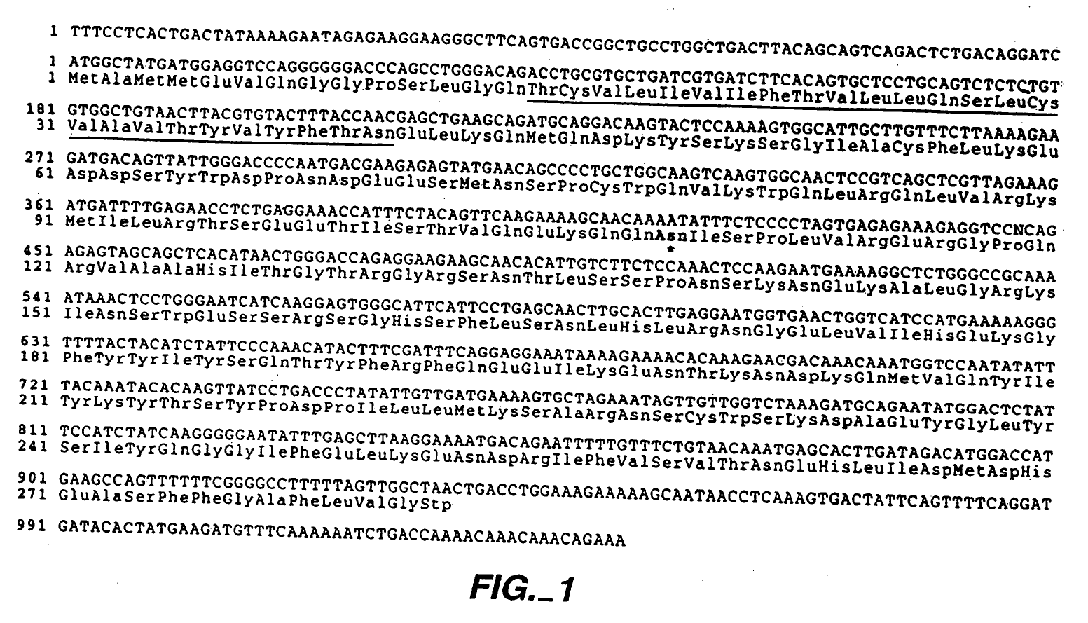

The terms “Apo-2 ligand”, “Apo-2L”, “Apo2L”, “Apo2L/TRAIL”, “Apo-2 ligand/TRAIL” and “TRAIL” are used herein interchangeably to refer to a polypeptide sequence which includes amino acid residues 114-281, inclusive, 95-281, inclusive, residues 92-281, inclusive, residues 91-281, inclusive, residues 41-281, inclusive, residues 39-281, inclusive, residues 15-281, inclusive, or residues 1-281, inclusive, of the amino acid sequence shown in FIG. 1 (SEQ ID NO:1), as well as biologically active fragments, deletional, insertional, or substitutional variants of the above sequences. In one embodiment, the polypeptide sequence comprises residues 114-281 of FIG. 1 (SEQ ID NO:1). Optionally, the polypeptide sequence comprises residues 92-281 or residues 91-281 of FIG. 1 (SEQ ID NO:1). The Apo-2L polypeptides may be encoded by the native nucleotide sequence shown in FIG. 1 (SEQ ID NO:2). Optionally, the codon which encodes residue Proll9 (FIG. 1; SEQ ID NO:2) may be “CCT” or “CCG”. Optionally, the fragments or variants are biologically active and have at least about 80% amino acid sequence identity, more preferably at least about 90% sequence identity, and even more preferably, at least 95%, 96%, 97%, 98%, or 99% sequence identity with any one of the above sequences. The definition encompasses substitutional variants of Apo-2 ligand in which at least one of its native amino acids are substituted by another amino acid such as an alanine residue. Optional variants may comprise an amino acid sequence which differs from the native sequence Apo-2 ligand polypeptide sequence of FIG. 1 (SEQ ID NO:1) and has one or more of the following amino acid substitutions at the residue position(s) in FIG. 1 (SEQ ID NO:1): S96C; S101C; S111C; R170C; K179C. The definition also encompasses a native sequence Apo-2 ligand isolated from an Apo-2 ligand source or prepared by recombinant or synthetic methods. The Apo-2 ligand of the invention includes the polypeptides referred to as Apo-2 ligand or TRAIL disclosed in WO97/01633 published Jan. 16, 1997, WO97/25428 published Jul. 17, 1997, WO99/36535 published Jul. 22, 1999, WO 01/00832 published Jan. 4, 2001, WO02/09755 published Feb. 7, 2002, and WO 00/75191 published Dec. 14, 2000. The terms are used to refer generally to forms of the Apo-2 ligand which include monomer, dimer, trimer, hexamer or hight oligomer forms of the polypeptide. All numbering of amino acid residues referred to in the Apo-2L sequence use the numbering according to FIG. 1 (SEQ ID NO:1), unless specifically stated otherwise. For instance, “D203” or “Asp203” refers to the aspartic acid residue at position 203 in the sequence provided in FIG. 1 (SEQ ID NO:1).

The term “Apo-2 ligand selective variant” as used herein refers to an Apo-2 ligand polypeptide which includes one or more amino acid mutations in a native Apo-2 ligand sequence and has selective binding affinity for either the DR4 receptor or the DR5 receptor. In one embodiment, the Apo-2 ligand variant has a selective binding affinity for the DR4 receptor and includes one or more amino acid substitutions in any one of positions 189, 191, 193, 199, 201 or 209 of a native Apo-2 ligand sequence. In another embodiment, the Apo-2 ligand variant has a selective binding affinity for the DR5 receptor and includes one or more amino acid substitutions in any one of positions 189, 191, 193, 264, 266, 267 or 269 of a native Apo-2 ligand sequence.

Preferred Apo-2 ligand selective variants include one or more amino acid mutations and exhibit binding affinity to the DR4 receptor which is equal to or greater (≧) than the binding affinity of native sequence Apo-2 ligand to the DR4 receptor, and even more preferably, the Apo-2 ligand variants exhibit less binding affinity (<) to the DR5 receptor than the binding affinity exhibited by native sequence Apo-2 ligand to DR5. When binding affinity of such Apo-2 ligand variant to the DR4 receptor is approximately equal (unchanged) or greater than (increased) as compared to native sequence Apo-2 ligand, and the binding affinity of the Apo-2 ligand variant to the DR5 receptor is less than or nearly eliminated as compared to native sequence Apo-2 ligand, the binding affinity of the Apo-2 ligand variant, for purposes herein, is considered “selective” for the DR4 receptor. Preferred DR4 selective Apo-2 ligand variants of the invention will have at least 10-fold less binding affinity to DR5 receptor (as compared to native sequence Apo-2 ligand), and even more preferably, will have at least 100-fold less binding affinity to DR5 receptor (as compared to native sequence Apo-2 ligand). The respective binding affinity of the Apo-2 ligand variant may be determined and compared to the binding properties of native Apo-2L (such as the 114-281 form) by ELISA, RIA, and/or BIAcore assays, known in the art. Preferred DR4 selective Apo-2 ligand variants of the invention will induce apoptosis in at least one type of mammalian cell (preferably a cancer cell), and such apoptotic activity can be determined by known art methods such as the alamar blue or crystal violet assay. The DR4 selective Apo-2 ligand variants may or may not have altered binding affinities to any of the decoy receptors for Apo-2L, those decoy receptors being referred to in the art as DcRl, DcR2 and OPG.

Further preferred Apo-2 ligand selective variants include one or more amino acid mutations and exhibit binding affinity to the DR5 receptor which is equal to or greater (≧) than the binding affinity of native sequence Apo-2 ligand to the DR5 receptor, and even more preferably, such Apo-2 ligand variants exhibit less binding affinity (<) to the DR4 receptor than the binding affinity exhibited by native sequence Apo-2 ligand to DR4. When binding affinity of such Apo-2 ligand variant to the DR5 receptor is approximately equal (unchanged) or greater than (increased) as compared to native sequence Apo-2 ligand, and the binding affinity of the Apo-2 ligand variant to the DR4 receptor is less than or nearly eliminated as compared to native sequence Apo-2 ligand, the binding affinity of the Apo-2 ligand variant, for purposes herein, is considered “selective” for the DR5 receptor. Preferred DR5 selective Apo-2 ligand variants of the invention will have at least 10-fold less binding affinity to DR4 receptor (as compared to native sequence Apo-2 ligand), and even more preferably, will have at least 100-fold less binding affinity to DR4 receptor (as compared to native sequence Apo-2 ligand). The respective binding affinity of the Apo-2 ligand variant may be determined and compared to the binding properties of native Apo2L (such as the 114-281 form) by ELISA, RIA, and/or BIAcore assays, known in the art. Preferred DR5 selective Apo-2 ligand variants of the invention will induce apoptosis in at least one type of mammalian cell (preferably a cancer cell), and such apoptotic activity can be determined by known art methods such as the alamar blue or crystal violet assay. The DR5 selective Apo-2 ligand variants may or may not have altered binding affinities to any of the decoy receptors for Apo-2L, those decoy receptors being referred to in the art as DcR1, DcR2 and OPG.

For purposes of shorthand designation of Apo-2 ligand variants described herein, it is noted that numbers refer to the amino acid residue position along the amino acid sequence of the putative native Apo-2 ligand (see FIG. 1).

Amino acid identification herein uses the single-letter alphabet of amino acids, i.e.,

| | | | | | |
|---|---|---|---|---|---|
| Asp | D | Aspartic acid | Ile | I | Isoleucine |
| Thr | T | Threonine | Leu | L | Leucine |
| Ser | S | Serine | Tyr | Y | Tyrosine |
| Glu | E | Glutamic acid | Phe | F | Phenylalanine |
| Pro | P | Proline | His | H | Histidine |
| Gly | G | Glycine | Lys | K | Lysine |
| Ala | A | Alanine | Arg | R | Arginine |
| Cys | C | Cysteine | Trp | W | Tryptophan |
| Val | V | Valine | Gln | Q | Glutamine |
| Met | M | Methionine | Asn | N | Asparagine |

“Death receptor antibody” is used herein to refer generally to antibody or antibodies directed to a receptor in the tumor necrosis factor receptor superfamily and containing a death domain capable of signalling apoptosis, and such antibodies include DR5 antibody and DR4 antibody.

“DR5 receptor antibody”, “DR5 antibody”, or “anti-DR5 antibody” is used in a broad sense to refer to antibodies that bind to at least one form of a DR5 receptor or extracellular domain thereof. Optionally the DR5 antibody is fused or linked to a heterologous sequence or molecule. Preferably the heterologous sequence allows or assists the antibody to form higher order or oligomeric complexes. Optionally, the DR5 antibody binds to DR5 receptor but does not bind or cross-react with any additional Apo-2L receptor (e.g. DR4, DcR1, or DcR2). Optionally the antibody is an agonist of DRS signalling activity.

Optionally, the DR5 antibody of the invention binds to a DR5 receptor at a concentration range of about 0.1 nM to about 20 mM as measured in a BIAcore binding assay. Optionally, the DR5 antibodies of the invention exhibit an Ic 50 value of about 0.6 nM to about 18 mM as measured in a BIAcore binding assay..

“DR4 receptor antibody”, “DR4 antibody”, or “anti-DR4 antibody” is used in a broad sense to refer to antibodies that bind to at least one form of a DR4 receptor or extracellular domain therof. Optionally the DR4 antibody is fused or linked to a heterologous sequence or molecule. Preferably the heterologous sequence allows or assists the antibody to form higher order or oligomeric complexes. Optionally, the DR4 antibody binds to DR4 receptor but does not bind or cross-react with any additional Apo-2L receptor (e.g. DR5, DcR1, or DcR2). Optionally the antibody is an agonist of DR4 signalling activity.

Optionally, the DR4 antibody of the invention binds to a DR4 receptor at a concentration range of about 0.1 nM to about 20 mM as measured in a BIAcore binding assay. Optionally, the DR5 antibodies of the invention exhibit an Ic 50 value of about 0.6 nM to about 18 mM as measured in a BIAcore binding assay.

The term “agonist” is used in the broadest sense, and includes any molecule that partially or fully enhances, stimulates or activates one or more biological activities of Apo2L/TRAIL, DR4 or DR5, in vitro, in situ, or in vivo. Examples of such biological activities binding of Apo2L/TRAIL to DR4 or DR5, include apoptosis as well as those further reported in the literature. An agonist may function in a direct or indirect manner. For instance, the agonist may function to partially or fully enhance, stimulate or activate one or more biological activities of DR4 or DR5, in vitro, in situ, or in vivo as a result of its direct binding to DR4 or DR5, which causes receptor activation or signal transduction. The agonist may also function indirectly to partially or fully enhance, stimulate or activate one or more biological activities of DR4 or DR5, in vitro, in situ, or in vivo as a result of, e.g., stimulating another effector molecule which then causes DR4 or DR5 activation or signal transduction. It is contemplated that an agonist may act as an enhancer molecule which functions indirectly to enhance or increase DR4 or DR5 activation or activity. For instance, the agonist may enhance activity of endogenous Apo-2L in a mammal. This could be accomplished, for example, by pre-complexing DR4 or DR5 or by stabilizing complexes of the respective ligand with the DR4 or DR5 receptor (such as stabilizing native complex formed between Apo-2L and DR4 or DR5).

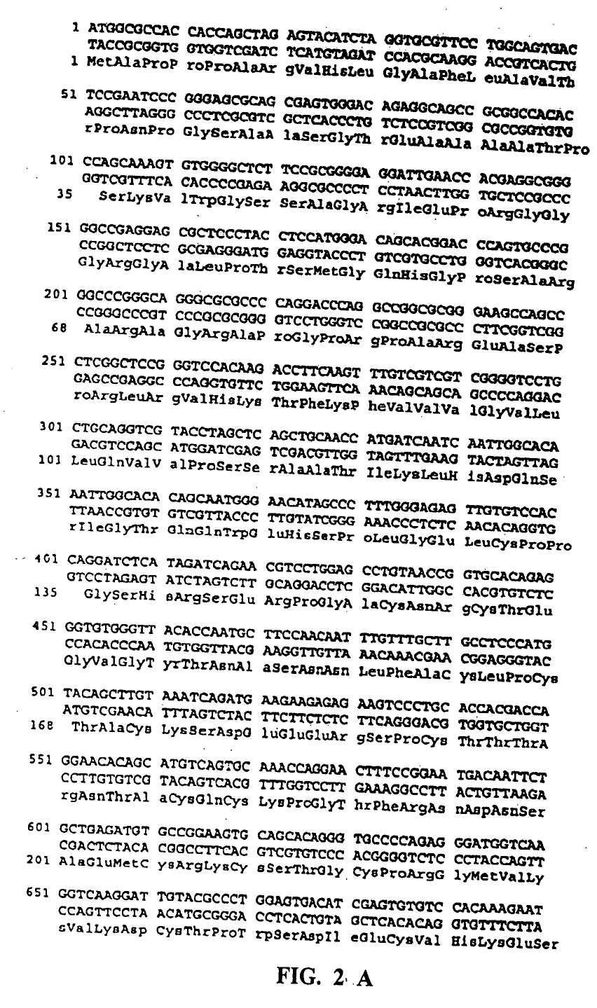

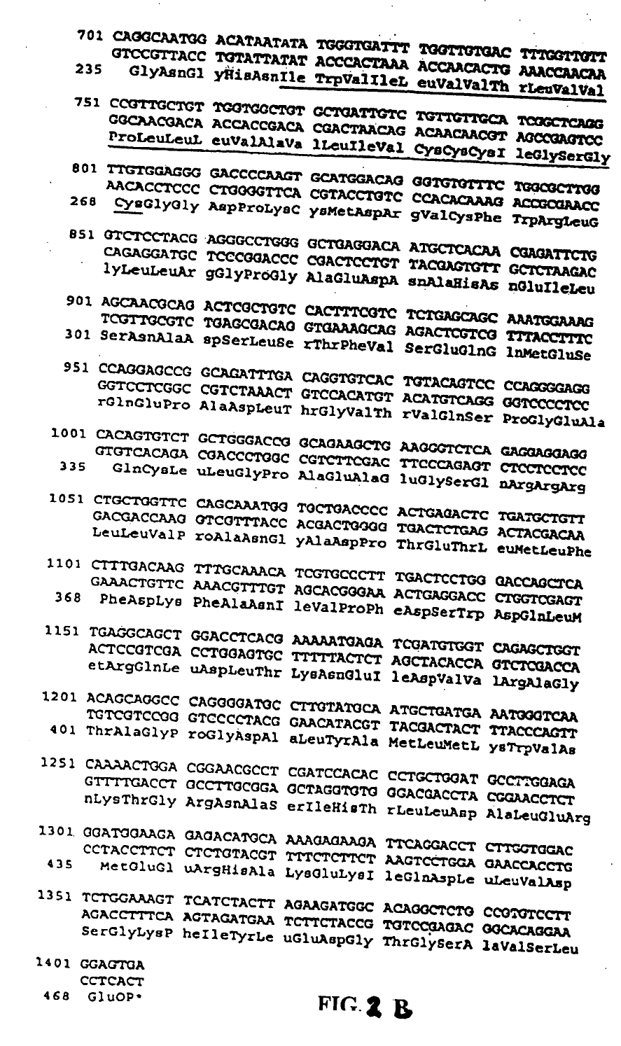

The term “DR4” and “DR4 receptor” as used herein refers to full length and soluble, extracellular domain forms of the receptor described in Pan et al., Science, 276:111-113 (1997); WO98/32856 published Jul. 30, 1998; U.S. Pat. No. 6,342,363 issued Jan. 29, 2002; and WO99/37684 published Jul. 29, 1999. The full length amino acid sequence of DR4 receptor is provided herein in FIG. 2.

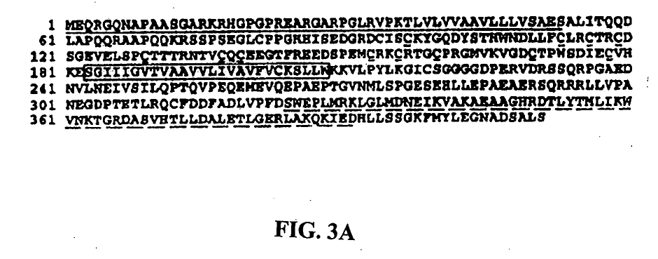

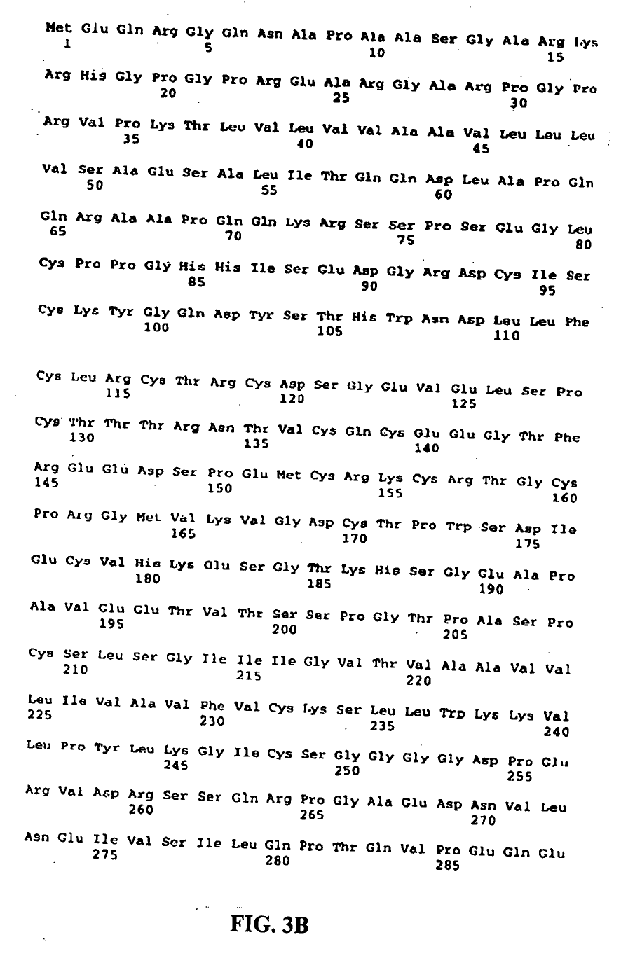

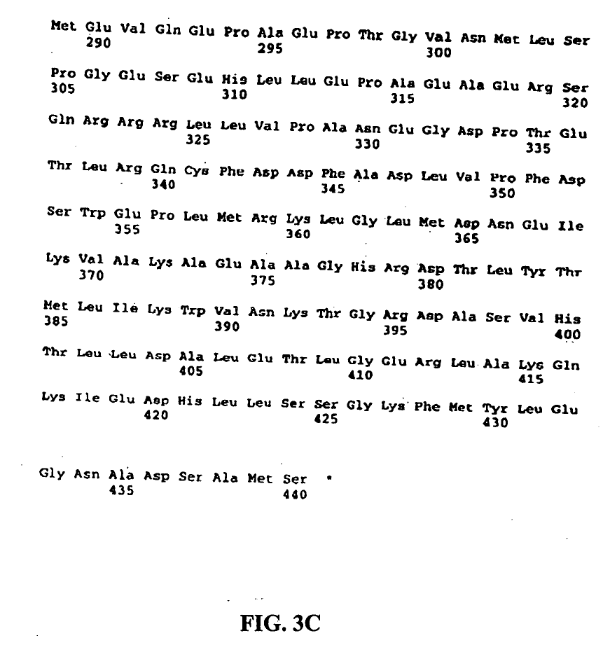

The term “DR5” and “DR5 receptor” as used herein refers to the full length and soluble, extracellular domain forms of the receptor described in Sheridan et al., Science, 277:818-821 (1997); Pan et al., Science, 277:815-818 (1997), U.S. Pat. No. 6,072,047 issued Jun. 6, 2000; U.S. Pat. No. 6,342,369, WO98/51793 published Nov. 19, 1998; WO98/41629 published Sep. 24, 1998; Screaton et al., Curr. Biol., 7:693-696 (1997); Walczak et al., EMBO J., 16:5386-5387 (1997); Wu et al., Nature Genetics, 17:141-143 (1997); WO98/35986 published Aug. 20, 1998; EP870,827 published Oct. 14, 1998; WO98/46643 published Oct. 22, 1998; WO99/02653 published Jan. 21, 1999; WO99/09165 published Feb. 25, 1999; WO99/11791 published Mar. 11, 1999. The DR5 receptor has also been referred to in the art as Apo-2; TRAIL-R, TR6, Tango-63, hAPO8, TRICK2 or KILLER. The term DR5 receptor used herein includes the full length 411 amino acid polypeptide provided in FIG. 3A and the full length 440 amino acid polypeptide provided in FIGS. 3B-C.

The term “polyol” when used herein refers broadly to polyhydric alcohol compounds. Polyols can be any water-soluble poly(alkylene oxide) polymer for example, and can have a linear or branched chain. Preferred polyols include those substituted at one or more hydroxyl positions with a chemical group, such as an alkyl group having between one and four carbons. Typically, the polyol is a poly(alkylene glycol), preferably poly(ethylene glycol) (PEG). However, those skilled in the art recognize that other polyols, such as, for example, poly(propylene glycol) and polyethylene-polypropylene glycol copolymers, can be employed using the techniques for conjugation described herein for PEG. The polyols of the invention include those well known in the art and those publicly available, such as from commercially available sources.

The term “conjugate” is used herein according to its broadest definition to mean joined or linked together. Molecules are “conjugated” when they act or operate as if joined.

The term “extracellular domain” or “ECD” refers to a form of ligand or receptor which is essentially free of transmembrane and cytoplasmic domains. Ordinarily, the soluble ECD will have less than 1% of such transmembrane and cytoplasmic domains, and preferably, will have less than 0.5% of such domains.

The term “divalent metal ion” refers to a metal ion having two positive charges. Examples of divalent metal ions for use in the present invention include but are not limited to zinc, cobalt, nickel, cadmium, magnesium, and manganese. Particular forms of such metals that may be employed include salt forms (e.g., pharmaceutically acceptable salt forms), such as chloride, acetate, carbonate, citrate and sulfate forms of the above mentioned divalent metal ions. A preferred divalent metal ion for use in the present invention is zinc, and more preferably, the salt form, zinc sulfate. Divalent metal ions, as described herein, are preferably employed in concentrations or amounts (e.g., effective amounts) which are sufficient to, for example, (1) enhance storage stability of Apo-2L trimers over a desired period of time, (2) enhance production or yield of Apo-2L trimers in a recombinant cell culture or purification method, (3) enhance solubility (or reduce aggregation) of Apo-2L trimers, or (4) enhance Apo-2L trimer formation.

“Isolated,” when used to describe the various proteins disclosed herein, means protein that has been identified and separated and/or recovered from a component of its natural environment. Contaminant components of its natural environment are materials that would typically interfere with diagnostic or therapeutic uses for the protein, and may include enzymes, hormones, and other proteinaceous or non-proteinaceous solutes. In preferred embodiments, the protein will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated protein includes protein in situ within recombinant cells, since at least one component of the Apo-2 ligand natural environment will not be present. Ordinarily, however, isolated protein will be prepared by at least one purification step.

An “isolated” nucleic acid molecule is a nucleic acid molecule that is identified and separated from at least one contaminant nucleic acid molecule with which it is ordinarily associated in the natural source of the nucleic acid. An isolated Apo-2 ligand nucleic acid molecule is other than in the form or setting in which it is found in nature. Isolated Apo-2 ligand nucleic acid molecules therefore are distinguished from the Apo-2 ligand nucleic acid molecule as it exists in natural cells. However, an isolated Apo-2 ligand nucleic acid molecule includes Apo-2 ligand nucleic acid molecules contained in cells that ordinarily express Apo-2 ligand where, for example, the nucleic acid molecule is in a chromosomal location different from that of natural cells.

“Percent (%) amino acid sequence identity” with respect to the sequences identified herein is defined as the percentage of amino acid residues in a candidate sequence that are identical with the amino acid residues in the Apo-2 ligand sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art can determine appropriate parameters for measuring alignment, including assigning algorithms needed to achieve maximal alignment over the full-length sequences being compared. For purposes herein, percent amino acid identity values can be obtained using the sequence comparison computer program, ALIGN-2, which was authored by Genentech, Inc. and the source code of which has been filed with user documentation in the US Copyright Office, Washington, DC, 20559, registered under the US Copyright Registration No. TXU510087. The ALIGN-2 program is publicly available through Genentech, Inc., South San Francisco, Calif. All sequence comparison parameters are set by the ALIGN-2 program and do not vary.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcTRIII and carry out ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)). FcRs herein include polymorphisms such as the genetic dimorphism in the gene that encodes FcγRIIIa resulting in either a phenylalanine (F) or a valine (V) at amino acid position 158, located in the region of the receptor that binds to IgG1. The homozygous valine FcγRIIIa (FcγRIIIa-158V) has been shown to have a higher affinity for human IgG1 and mediate increased ADCC in vitro relative to homozygous phenylalanine FcγRIIIa (FcγRIIIa-158F) or heterozygous (FcγRIIIa-158F/V) receptors.

“Complement dependent cytotoxicity” or “CDC” refer to the ability of a molecule to lyse a target in the presence of complement. The complement activation pathway is initiated by the binding of the first component of the complement system (Clq) to a molecule (e.g. an antibody) complexed with a cognate antigen. To assess complement activation, a CDC assay, e.g. as described in Gazzano-Santoro et al., J. Immunol. Methods 202:163 (1996), may be performed.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cell-mediated cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody which are responsible for antigen-binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

An antibody “which binds” an antigen of interest, e.g. a death receptor such as DR4 or DR5, is one capable of binding that antigen with sufficient affinity and/or avidity such that the antibody is useful as a therapeutic agent for targeting a cell expressing the antigen.

For the purposes herein, “immunotherapy” will refer to a method of treating a mammal (preferably a human patient) with an antibody, wherein the antibody may be an unconjugated or “naked” antibody, or the antibody may be conjugated or fused with heterologous molecule(s) or agent(s), such as one or more cytotoxic agent(s), thereby generating an “immunoconjugate”.

An “isolated” antibody is one which has been identified and separated and/or recovered from a component of its natural environment. Contaminant components of its natural environment are materials which would interfere with diagnostic or therapeutic uses for the antagonist or antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The expression “effective amount” refers to an amount of the combination of a death receptor agonist and an EGFR inhibitor which is effective for preventing, ameliorating or treating the disease or condition in question whether administered simultaneously or sequentially. In particular embodiments, an effective amount is the amount of the death receptor agonist and EGFR inhibitor combination sufficient to enhance, or otherwise increase the propensity (such as synergistically) of a cell to undergo apoptosis, reduce tumour volume, or prolong survival of a mammal having a cancer.

The term “immunosuppressive agent” as used herein for adjunct therapy refers to substances that act to suppress or mask the immune system of the mammal being treated herein. This would include substances that suppress cytokine production, downregulate or suppress self-antigen expression, or mask the MHC antigens. Examples of such agents include 2-amino-6-aryl-5-substituted pyrimidines (see U.S. Pat. No. 4,665,077; nonsteroidal antiinflammatory drugs (NSAIDs); azathioprine; cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as glucocorticosteroids, e.g., prednisone, methylprednisolone, dexamethasone, and hydrocortisone; methotrexate (oral or subcutaneous); hydroxycloroquine; sulfasalazine; leflunomide; cytokine or cytokine receptor antagonists including anti-interferon-γ, -β, or -α antibodies, anti-tumor necrosis factor-α antibodies (infliximab or adalimumab), anti-TNFα immunoahesin (etanercept), anti-tumor necrosis factor-β antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (wo 90/08187 published Jul. 26, 1990); streptokinase; TGF-β; streptodornase; RNA or DNA from the host; FK506; RS-61443; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); and T cell receptor antibodies (EP 340,109) such as T10B9.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, or fragments thereof.

“Synergistic activity” or “synergy” or “synergistic effect” or “synergistic effective amount” for the purposes herein means that the effect observed when employing a combination of Apo2L/TRAIL or death receptor antibody and EGFR inhibitor is (1) greater than the effect achieved when that Apo2L/TRAIL, death receptor antibody or EGFR inhibitor is employed alone (or individually) and (2) greater than the sum added (additive) effect for that Apo2L/TRAIL or death receptor antibody and EGFR inhibitor. Such synergy or synergistic effect can be determined by way of a variety of means known to those in the art. For example, the synergistic effect of Apo2L/TRAIL or death receptor antibody and EGFR inhibitor can be observed in in vitro or in vivo assay formats examining reduction of tumor cell number or tumor mass.

The terms “apoptosis” and “apoptotic activity” are used in a broad sense and refer to the orderly or controlled form of cell death in mammals that is typically accompanied by one or more characteristic cell changes, including condensation of cytoplasm, loss of plasma membrane microvilli, segmentation of the nucleus, degradation of chromosomal DNA or loss of mitochondrial function. This activity can be determined and measured using well known art methods, for instance, by cell viability assays, FACS analysis or DNA electrophoresis, binding of annexin V, fragmentation of DNA, cell shrinkage, dilation of endoplasmic reticulum, cell fragmentation, and/or formation of membrane vesicles (called apoptotic bodies).

The terms “cancer”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer (NSCLC), gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer, colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer.

The term “immune related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are autoimmune diseases, immune-mediated inflammatory diseases, non-immune-mediated inflammatory diseases, infectious diseases, and immunodeficiency diseases. Examples of immune-related and inflammatory diseases, some of which are immune or T cell mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjogren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barre syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory and fibrotic lung diseases such as inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases include AIDS (HIV infection), hepatitis A, B, C, D, and E, bacterial infections, fungal infections, protozoal infections and parasitic infections.

A “B cell malignancy” is a malignancy involving B cells. Examples include Hodgkin's disease, including lymphocyte predominant Hodgkin's disease (LPHD); non-Hodgkin's lymphoma (NHL); follicular center cell (FCC) lymphoma; acute lymphocytic leukemia (ALL); chronic lymphocytic leukemia (CLL); hairy cell leukemia; plasmacytoid lymphocytic lymphoma; mantle cell lymphoma; AIDS or HIV-related lymphoma; multiple myeloma; central nervous system (CNS) lymphoma; post-transplant lymphoproliferative disorder (PTLD); Waldenstrom's macroglobulinemia (lymphoplasmacytic lymphoma); mucosa-associated lymphoid tissue (MALT) lymphoma; and marginal zone lymphoma/leukemia.

Non-Hodgkin's lymphoma (NHL) includes, but is not limited to, low grade/follicular NHL, relapsed or refractory NHL, front line low grade NHL, Stage III/IV NHL, chemotherapy resistant NHL, small lymphocytic (SL) NHL, intermediate grade/follicular NHL, intermediate grade diffuse NHL, diffuse large cell lymphoma, aggressive NHL (including aggressive front-line NHL and aggressive relapsed NHL), NHL relapsing after or refractory to autologous stem cell transplantation, high grade immunoblastic NHL, high grade lymphoblastic NHL, high grade small non-cleaved cell NHL, bulky disease NHL, etc.

An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis, juvenile rheumatoid arthritis, osteoarthritis, psoriatic arthritis, and ankylosing spondylitis), psoriasis, dermatitis including atopic dermatitis; chronic idiopathic urticaria, including chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), and IBD with co-segregate of pyoderma gangrenosum, erythema nodosum, primary sclerosing cholangitis, and/or episcleritis), respiratory distress syndrome, including adult respiratory distress syndrome (ARDS), meningitis, IgE-mediated diseases such as anaphylaxis and allergic rhinitis, encephalitis such as Rasmussen's encephalitis, uveitis, colitis such as microscopic colitis and collagenous colitis, glomerulonephritis (GN) such as membranous GN, idiopathic membranous GN, membranous proliferative GN (MPGN), including Type I and Type II, and rapidly progressive GN, allergic conditions, eczema, asthma, conditions involving infiltration of T cells and chronic inflammatory responses, atherosclerosis, autoimmune myocarditis, leukocyte adhesion deficiency, systemic lupus erythematosus (SLE) such as cutaneous SLE, lupus (including nephritis, cerebritis, pediatric, non-renal, discoid, alopecia), juvenile onset diabetes, multiple sclerosis (MS) such as spino-optical MS, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including Large Vessel vasculitis (including Polymyalgia Rheumatica and Giant Cell (Takayasu's) Arteritis), Medium Vessel vasculitis (including Kawasaki's Disease and Polyarteritis Nodosa), CNS vasculitis, and ANCA-associated vasculitis , such as Churg-Strauss vasculitis or syndrome (CSS)), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, and rejection arising from renal transplantation, liver transplantation, intestinal transplantation, cardiac transplantation, etc.), graft versus host disease (GVHD), pemphigoid bullous, pemphigus (including vulgaris, foliaceus, and pemphigus mucus-membrane pemphigoid), autoimmune polyendocrinopathies, Reiter's disease, stiff-man syndrome, immune complex nephritis, IgM polyneuropathies or IgM mediated neuropathy, idiopathic thrombocytopenic purpura (ITP), thrombotic throbocytopenic purpura (TTP), thrombocytopenia (as developed by myocardial infarction patients, for example), including autoimmune thrombocytopenia, autoimmune disease of the testis and ovary including autoimune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM), including pediatric IDDM, and Sheehan's syndrome; autoimmune hepatitis, Lymphoid interstitial pneumonitis (HIV), bronchiolitis obliterans (non-transplant) vs NSIP, Guillain-Barre Syndrome, Berger's Disease (IgA nephropathy), primary biliary cirrhosis, celiac sprue (gluten enteropathy), refractory sprue with co-segregate dermatitis herpetiformis, cryoglobulinemia, amylotrophic lateral sclerosis (ALS; Lou Gehrig's disease), coronary artery disease, autoimmune inner ear disease (AIED), autoimmune hearing loss, opsoclonus myoclonus syndrome (OMS), polychondritis such as refractory polychondritis, pulmonary alveolar proteinosis, amyloidosis, giant cell hepatitis, scleritis, monoclonal gammopathy of uncertain/unknown significance (MGUS), peripheral neuropathy, paraneoplastic syndrome, channelopathies such as epilepsy, migraine, arrhythmia, muscular disorders, deafness, blindness, periodic paralysis, and channelopathies of the CNS; autism, inflammatory myopathy, and focal segmental glomerulosclerosis (FSGS).

The term “prodrug” as used in this application refers to a precursor or derivative form of a pharmaceutically active substance that is less cytotoxic to cancer cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). Prodrugs also refer to percursors or derivatives of a parent pharmaceutically active substance that has greater bioavailability than the parent substance and is converted to the parent compound in vivo. For example, a prodrug may have greater absorption into the bloodstream upon oral ingestion compared to the parent substance. The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described below.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammalI and calicheamicin omegal1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCINO doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,22″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Illinois), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON- toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “growth inhibitory agent” when used herein refers to a compound or composition which inhibits growth of a cell, either in vitro or in vivo. Thus, the growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (WB Saunders: Philadelphia, 1995), especially p. 13.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors; platelet-growth factor; transforming growth factors (TGFs) such as TGF-α and TGF-β; insulin-like growth factor-I and -II; erythropoietin (EPO); osteoinductive factors; interferons such as interferon-α, -β, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-2, IL-3, IL-4, IL-5, IL-6, IL-7, IL-8, IL-9, IL-11, IL-12; and other polypeptide factors including LIF and kit ligand (KL). As used herein, the term cytokine includes proteins from natural sources or from recombinant cell culture and biologically active equivalents of the native sequence cytokines.

The terms “treating”, “treatment” and “therapy” as used herein refer to curative therapy, prophylactic therapy, and preventative therapy.

The term “mammal” as used herein refers to any mammal classified as a mammal, including humans, cows, horses, dogs and cats. In a preferred embodiment of the invention, the mammal is a human.

The term “EGFR” refers to the receptor tyrosine kinase polypeptide Epidermal Growth Factor Receptor which is described in Ullrich et al, Nature (1984) 309:418425, alternatively referred to as Her-1 and the c-erbB gene product, as well as variants thereof such as EGFRvIII. Variants of EGFR also include deletional, substitutional and insertional variants, for example those described in Lynch et al (New England Journal of Medicine 2004, 350:2129), Paez et al (Science 2004, 304:1497), Pao et al (PNAS 2004, 101:13306).

The term “EGFR inhibitor” refers to compounds that bind to or otherwise interact directly with EGFR and prevent or reduce its signalling activity, and is alternatively referred to as an “EGFR antagonist”. EGFR inhibitor include antibodies such as chimeric antibody C225 also referred to as cetuximab and Erbitux® (ImClone Systems Inc.), fully human ABX-EGF (panitumumab, Abgenix Inc.), as well as fully human antibodies known as E1.1, E2.4, E2.5, E6.2, E6.4, E2.11, E6.3 and E7.6.3 and described in U.S. Pat. No. 6,235,883; MDX-447 (Medarex Inc). EGFR antagonists include small molecules such as compounds described in U.S. Pat. Nos. 5,616,582, 5,457,105, 5,475,001, 5,565,4307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, WO9814451, WO9850038, WO9909016, WO9924037, U.S. Pat. Nos. 6,344,455, 5,760,041, 6,002,008, 5,747,498. Particular small molecule EGFR antagonsists include OSI-774 (CP-358774, erlotinib, OSI Pharmaceuticals); PD 183805 (CI 1033, 2-propenamide, N-[4-[(3-chloro-4-fluorophenyl)amino)-7-[3-(4-morpholinyl)propoxy]-6-quinazolinyl]-, dihydrochloride, Pfizer Inc.); Iressa (ZD1839, gefitinib, 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl)-2-butynamide); and EKB-569 (N-[4-[(3-chloro-4-fluorophenyl)amino]-3-cyano-7-ethoxy-6-quinolinyl]-4-(dimethylamino)-2-butenamide).

In a particular embodiment, the EGFR inhibitor has a general formula I: 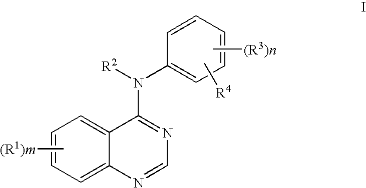
in accordance with U.S. Pat. No. 5,757,498 wherein:
X is halo or hydroxy;
m is 1, 2, or 3;
each R¹ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH;
or each R¹ is independently selected from R⁹ and C₁-C₄ alkyl substituted by cyano, wherein R⁹ is selected from the group consisting of R⁵, —OR⁶, —NR⁵ R⁶, —C(O)R⁷, —NHOR⁹, —OC(O)R⁶, cyano, A and —YR⁹; R⁵ is C₁-C₄ alkyl; R⁶ is independently hydrogen or R⁹; R⁷ is R⁹, —OR⁶ or —NR⁶R⁶ ; A is selected from piperidino, morpholino, pyrrolidino, 4-R⁶-piperazin-1-yl, imidazol-1-yl, 4-pyridon-1-yl, —(C₁-C₄ alkylene) (CO2H), phenoxy, phenyl, phenylsulfanyl, C₂-C₄ alkenyl, and —(C₁-C₄ alkylene)C(O)NR⁶R⁶; and Y is S, SO, or SO₂; wherein the alkyl moieties in R⁵, —OR⁶ and —NR⁶R⁶ are optionally substituted by one to three halo substituents and the alkyl moieties in R⁹, —OR⁶ and —NR⁶R⁶ are optionally substituted by 1 or 2 R⁹ groups, and wherein the alkyl moieties of said optional substituents are optionally substituted by halo or R⁹, with the proviso that two heteroatoms are not attached to the same carbon atom;
or each R¹ is independently selected from —NHSO₂R⁵, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄) -alkanoylamino wherein R¹⁰ is selected from halo, —OR⁶, C₂-C₄ alkanoyloxy, —C(O)R⁷, and —NR⁵R⁶; and wherein said —NHSO₂R⁵, phthalimido-(C₁-C₄-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino R¹ groups are optionally substituted by 1 or 2 substituents independently selected from halo, C₁-C₄ alkyl, cyano, methanesulfonyl and C₁-C₄ alkoxy;
or two R¹ groups are taken together with the carbons to which they are attached to form a 5-8 membered ring that includes 1 or 2 heteroatoms selected from O, S and N;
R² is hydrogen or C₁-C₆ alkyl optionally substituted by 1 to 3 substituents independently selected from halo, C₁-C₄ alkoxy, —NR⁶R⁶, and —SO₂R⁵ ;
n is 1 or 2 and each R³ is independently selected from hydrogen, halo, hydroxy, C₁-C₆ alkyl, —NR⁶R⁶, and C₁-C₄ alkoxy, wherein the alkyl moieties of said R³ groups are optionally substituted by 1 to 3 substituents independently selected from halo, C₁-C₄alkoxy, —NR⁶R⁶, and —SO₂R; and,
R⁴ is azido or -(ethynyl)-R¹¹ wherein R¹¹ is hydrogen or C₁-C₆ alkyl optionally substituted by hydroxy, —OR⁶, or —NR⁶R⁶.

In a particular embodiment, the EGFR inhibitor is a compound according to formula I selected from the group consisting of:
(6,7-dimethoxyquinazolin-4-yl)-(3-ethynylphenyl)-amine; (6,7-dimethoxyquinazolin-4-yl)-[3-(3′-hydroxypropyn-1-yl)phenyl]-amine; [3-(2′-(aminomethyl)-ethynyl)phenyl]-(6,7-dimethoxyquinazolin-4-yl)-amine; (3-ethynylphenyl)-(6-nitroquinazolin-4-yl)-amine; (6,7-dimethoxyquinazolin-4-yl)-(4-ethynylphenyl)-amine; (6,7-dimethoxyquinazolin-4-yl)-(3-ethynyl-2-methylphenyl)-amine; (6-aminoquinazolin-4-yl)-(3-ethynylphenyl)-amine; (3-ethynylphenyl)-(6-methanesulfonylaminoquinazolin-4-yl)-amine; (3-ethynylphenyl)-(6,7-methylenedioxyquinazolin-4-yl)-amine; (6,7-dimethoxyquinazolin-4-yl)-(3-ethynyl-6-methylphenyl)-amine; (3-ethynylphenyl)-(7-nitroquinazolin-4-yl)-amine; (3-ethynylphenyl)-[6-(4′-toluenesulfonylamino)quinazolin-4-yl]-amine; (3-ethynylphenyl)-{6-[2′-phthalimido-eth-1′-yl-sulfonylamino]quinazolin-4-yl}-amine; (3-ethynylphenyl)-(6-guanidinoquinazolin-4-yl)-amine; (7-aminoquinazolin-4-yl)-(3-ethynylphenyl)-amine; (3-ethynylphenyl)-(7-methoxyquinazolin-4-yl)-amine; (6-carbomethoxyquinazolin-4-yl)-(3-ethynylphenyl)-amine; (7-carbomethoxyquinazolin-4-yl)-(3-ethynylphenyl)-amine; [6,7-bis(2-methoxyethoxy)quinazolin-4-yl]-(3-ethynylphenyl)-amine; (3-azidophenyl)-(6,7-dimethoxyquinazolin-4-yl)-amine; (3-azido-5-chlorophenyl)-(6,7-dimethoxyquinazolin-4-yl)-amine; (4-azidophenyl)-(6,7-dimethoxyquinazolin-4-yl)-amine; (3-ethynylphenyl)-(6-methansulfonyl-quinazolin-4-yl)-amine; (6-ethansulfanyl-quinazolin-4-yl)-(3-ethynylphenyl)-amine; (6,7-dimethoxy-quinazolin-4-yl)-(3-ethynyl-4-fluoro-phenyl)-amine; (6,7-dimethoxy-quinazolin-4-yl)-[3-(propyn-1′-yl)-phenyl]-amine; [6,7-bis-(2-methoxy-ethoxy)-quinazolin-4-yl)-(5-ethynyl-2-methyl-phenyl)-amine; [6,7-bis-(2-methoxy-ethoxy)-quinazolin-4-yl]-(3-ethynyl-4-fluoro- phenyl)-amine; [6,7-bis-(2-chloro-ethoxy)-quinazolin-4-yl]-(3-ethynyl-phenyl)-amine; (6-(2-chloro-ethoxy)-7-(2-methoxy-ethoxy)-quinazolin-4-yl]-(3-ethynyl-phenyl)-amine; [6,7-bis-(2-acetoxy-ethoxy)-quinazolin-4-yl]-(3-ethynyl-phenyl)-amine; 2-[4-(3-ethynyl-phenylamino)-7-(2-hydroxy-ethoxy)-quinazolin-6-yloxy]-ethanol; [6-(2-acetoxy-ethoxy)-7-(2-methoxy-ethoxy)-quinazolin-4-yl]-(3-ethynyl-phenyl)-amine; [7-(2-chloro-ethoxy)-6-(2-methoxy-ethoxy)-quinazolin-4-yl]-(3-ethynyl-phenyl)-amine; [7-(2-acetoxy-ethoxy)-6-(2-methoxy-ethoxy)-quinazolin-4-yl]-(3-ethynyl-phenyl)-amine; 2-[4-(3-ethynyl-phenylamino)-6-(2-hydroxy-ethoxy)-quinazolin-7- yloxy]-ethanol; 2-[4-(3-ethynyl-phenylamino)-7-(2-methoxy-ethoxy)-quinazolin-6-yloxy]-ethanol; 2-[4-(3-ethynyl-phenylamino)-6-(2-methoxy-ethoxy)-quinazolin-7-yloxy]-ethanol; [6-(2-acetoxy-ethoxy)-7-(2-methoxy-ethoxy)-quinazolin-4-yl]-(3-ethynyl-phenyl)-amine; (3-ethynyl-phenyl)-{6-(2-methoxy-ethoxy)-7-(2-(4-methyl-piperazin-1-yl)-ethoxyl-quinazolin-4-yl}-amine; (3-ethynyl-phenyl)-[7-(2-methoxy-ethoxy)-6-(2-morpholin-4-yl)-ethoxy)-quinazolin-4-yl]-amine; (6,7-diethoxyquinazolin-1-yl)-(3-ethynylphenyl)-amine; (6,7-dibutoxyquinazolin-1-yl)-(3-ethynylphenyl)-amine; (6,7-diisopropoxyquinazolin-1-yl)-(3-ethynylphenyl)-amine; (6,7-diethoxyquinazolin-1-yl)-(3-ethynyl-2-methyl-phenyl)-amine; [6,7-bis-(2-methoxy-ethoxy)-quinazolin-1-yl]-(3-ethynyl-2-methyl-phenyl)-amine; (3-ethynylphenyl)-[6-(2-hydroxy-ethoxy)-7-(2-methoxy-ethoxy)-quinazolin-1-yl)-amine; [6,7-bis-(2-hydroxy-ethoxy)-quinazolin-1-yl]-(3-ethynylphenyl)-amine; 2-[4-(3-ethynyl-phenylamino)-6-(2-methoxy-ethoxy)-quinazolin-7-yloxy]-ethanol; (6,7-dipropoxy-quinazolin-4-yl)-(3-ethynyl-phenyl)-amine; (6,7-diethoxy-quinazolin-4-yl)-(3-ethynyl-5-fluoro-phenyl)-amine; (6,7-diethoxy-quinazolin-4-yl)-(3-ethynyl-4-fluoro-phenyl)-amine; (6,7-diethoxy-quinazolin-4-yl)-(5-ethynyl-2-methyl-phenyl)-amine; (6,7-diethoxy-quinazolin-4-yl)-(3-ethynyl-4-methyl-phenyl)-amine; (6-aminomethyl-7-methoxy-quinazolin-4-yl)-(3-ethynyl-phenyl)-amine; (6-aminomethyl-7-methoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-methoxy-quinazolin-4-yl)-(3-ethynylphenyl)-amine; (6-aminocarbonylmethyl-7-ethoxy-quinazolin-4-yl)-(3-ethynylphenyl)-amine; (6-aminocarbonylethyl-7-ethoxy-quinazolin-4-yl)-(3-ethynylphenyl)-amine; (6-aminocarbonylmethyl-7-isopropoxy-quinazolin-4-yl)-(3-ethynylphenyl)-amine; (6-aminocarbonylmethyl-7-propoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-isopropoxy-quinazolin-4-yl)-(3-ethynylphenyl)-amine; and (6-aminocarbonylethyl-7-propoxy-quinazolin-4-yl)-(3-ethynylphenyl)-amine; (6,7-diethoxyquinazolin-1-yl)-(3-ethynylphenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; [6,7-bis-(2-methoxy-ethoxy)-quinazolin-1-yl]-(3-ethynylphenyl)-amine; (6,7-dimethoxyquinazolin-1-yl)-(3-ethynylphenyl)-amine; (3-ethynylphenyl)-(6-methanesulfonylamino-quinazolin-1-yl)-amine; and (6-amino-quinazolin-1-yl)-(3-ethynylphenyl)-amine.

In a particular embodiment, the EGFR inhibitor of formula I is N-(3-ethynylphenyl)-6,7-bis(2-methoxyethoxy)-4-quinazolinamine. In a particular embodiment, the EGFR inhibitor N-(3-ethynylphenyl)-6,7-bis(2-methoxyethoxy)-4-quinazolinamine is an HCl salt form. In another particular embodiment, the EGFR inhibitor N-(3-ethynylphenyl)-6,7-bis(2-methoxyethoxy)-4-quinazolinamine is in a substantially homogeneous crystalline polymorph form (described as polymorph B in WO 01/34,574) that exhibits an X-ray powder diffraction pattern having characteristic peaks expressed in degrees 2-theta at approximately 6.26, 12.48, 13.39, 16.96, 20.20, 21.10, 22.98, 24.46, 25.14 and 26.91. Such polymorph form of N-(3-ethynylphenyl)-6,7-bis(2-methoxyethoxy)-4-quinazolinamine is referred to as Tarceva™ as well as OSI-774, CP-358774 and erlotinib.

The compounds of formula I, pharmaceutically acceptable salts and prodrugs thereof (hereafter the active compounds) may be prepared by any process known to be applicable to the preparation of chemically-related compounds. In general the active compounds may be made from the appropriately substituted quinazoline using the appropriately substituted amine as shown in the general scheme I disclosed in U.S. Pat. No. 5,747,498:

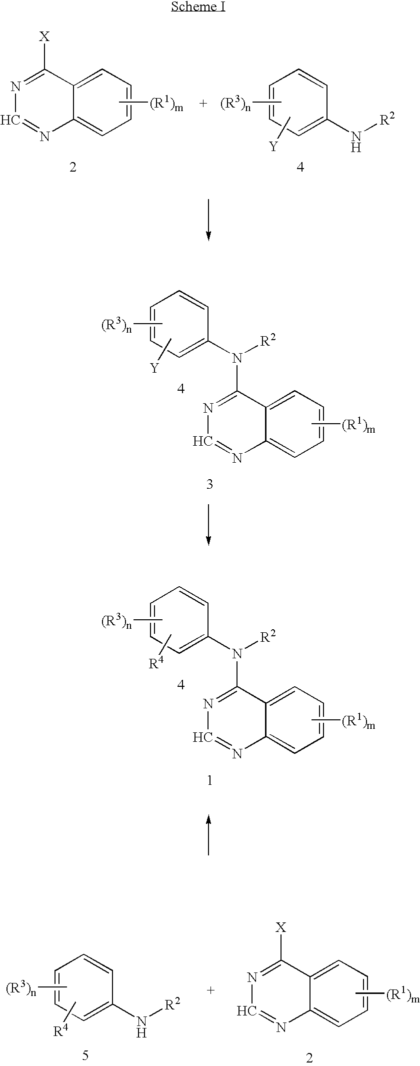

As shown in Scheme I the appropriate 4-substituted quinazoline 2 wherein X is a suitable displaceable leaving group such as halo, aryloxy, alkylsulfinyl, alkylsulfonyl such as trifluoromethanesulfonyloxy, arylsulfinyl, arylsulfonyl, siloxy, cyano, pyrazolo, triazolo or tetrazolo, preferably a 4-chloroquinazoline, is reacted with the appropriate amine or amine hydrochloride 4 or 5, wherein R⁴ is as described above and Y is Br, I, or trifluoromethane-sulfonyloxy in a solvent such as a (C₁-C₆)alcohol, dimethylformamide (DMF), N-methylpyrrolidin-2-one, chloroform, acetonitrile, tetrahydrofuran (THF), 1-4 dioxane, pyridine or other aprotic solvent. The reaction may be effected in the presence of a base, preferably an alkali or alkaline earth metal carbonate or hydroxide or a tertiary amine base, such as pyridine, 2,6-lutidine, collidine, N-methyl-morpholine, triethylamine, 4-dimethylamino-pyridine or N,N-dimethylaniline. These bases are hereinafter refered to as suitable bases. The reaction mixture is maintained at a temperature from about ambient to about the reflux temperature of the solvent, preferably from about 35° C. to about reflux, until substantially no remaining 4-haloquinazoline can be detected, typically about 2 to about 24 hours. Preferably, the reaction is performed under an inert atmosphere such as dry nitrogen.

Generally the reactants are combined stoichiometrically. When an amine base is used for those compounds where a salt (typically the HCl salt) of an amine 4 or 5 is used, it is preferable to use excess amine base, generally an extra equivalent of amine base. (Alternatively, if an amine base is not used an excess of the amine 4 or 5 may be used).

For those compounds where a sterically hindered amine 4 (such as a 2-alkyl-3-ethynylaniline) or very reactive 4-haloquinazoline is used it is preferable to use t-butyl alcohol or a polar aprotic solvent such as DMF or N-methylpyrrolidin-2-one as the solvent.

Alternatively, a 4-substituted quinazoline 2 wherein X is hydroxyl or oxo (and the 2-nitrogen is hydrogenated) is reacted with carbon tetrachloride and an optionally substituted triarylphosphine which is optionally supported on an inert polymer (e.g. triphenylphosphine, polymer supported, Aldrich Cat. No. 36,645-5, which is a 2% divinylbenzene cross-linked polystyrene containing 3 mmol phosphorous per gram resin) in a solvent such as carbon tetrachloride, chloroform, dichloroethane, tetrahydrofuran, acetonitrile or other aprotic solvent or mixtures thereof. The reaction mixture is maintained at a temperature from about ambient to reflux, preferably from about 35° C. to reflux, for 2 to 24 hours. This mixture is reacted with the appropriate amine or amine hydrochloride 4 or 5 either directly or after removal of solvent, for example by vacuum evaporation, and addition of a suitable alternative solvent such as a (C₁-C₆) alcohol, DMF, N-methylpyrrolidin-2-one, pyridine or 1-4 dioxane. Then, the reaction mixture is maintained at a temperature from about ambient to the reflux temperature of the solvent preferably from about 35° C. to about reflux, until substantially complete formation of product is acheived, typically from about 2 to about 24 hours. Preferably the reaction is performed under an inert atmosphere such as dry nitrogen.

When compound 4, wherein Y is Br, I, or trifluoromethanesulfonyloxy, is used as starting material in the reaction with quinazoline 2, a compound of formula 3 is formed wherein R¹, R², R³, and Y are as described above. Compound 3 is converted to compounds of formula 1 wherein R⁴ is R¹¹ ethynyl, and R¹¹ is as defined above, by reaction with a suitable palladium reagent such as tetrakis(triphenylphosphine)palladium or bis(triphenylphosphine)palladium dichloride in the presence of a suitable Lewis acid such as cuprous chloride and a suitable alkyne such as trimethylsilylacetylene, propargyl alcohol or 3-(N,N-dimethylamino)-propyne in a solvent such as diethylamine or triethylamine. Compounds 3, wherein Y is NH2, may be converted to compounds 1 wherein R⁴ is azide by treatment of compound 3 with a diazotizing agent, such as an acid and a nitrite (e.g., acetic acid and NaNO₂) followed by treatment of the resulting product with an azide, such as NaN₃.

For the production of those compounds of Formula I wherein an R¹ is an amino or hydroxyamino group the reduction of the corresponding Formula I compound wherein R¹ is nitro is employed.

The reduction may conveniently be carried out by any of the many procedures known for such transformations. The reduction may be carried out, for example, by hydrogenation of the nitro compound in a reaction-inert solvent in the presence of a suitable metal catalyst such as palladium, platinum or nickel. A further suitable reducing agent is, for example, an activated metal such as activated iron (produced by washing iron powder with a dilute solution of an acid such as hydrochloric acid). Thus, for example, the reduction may be carried out by heating a mixture of the nitro compound and the activated metal with concentrated hydrochloric acid in a solvent such as a mixture of water and an alcohol, for example, methanol or ethanol, to a temperature in the range, for example, 50° to 150° C., conveniently at or near 70° C. Another suitable class of reducing agents are the alkali metal dithionites, such as sodium dithionite, which may be used in (C₁-C₄)alkanoic acids, (C₁-C₆)alkanols, water or mixtures thereof.

For the production of those compounds of Formula I wherein R² or R³ incorporates a primary or secondary amino moiety (other than the amino group intended to react with the quinazoline), such free amino group is preferably protected prior to the above described reaction followed by deprotection, subsequent to the above described reaction with 4-(substituted)quinazoline 2.

Several well known nitrogen protecting groups can be used. Such groups include (C₁-C₆)alkoxycarbonyl, optionally substituted benzyloxycarbonyl, aryloxycarbonyl, trityl, vinyloxycarbonyl, o-nitrophenylsulfonyl, diphenylphosphinyl, p-toluenesulfonyl, and benzyl. The addition of the nitrogen protecting group may be carried out in a chlorinated hydrocarbon solvent such as methylene chloride or 1,2-dichloroethane, or an ethereal solvent such as glyme, diglyme or THF, in the presence or absence of a tertiary amine base such as triethylamine, diisopropylethylamine or pyridine, preferably triethylamine, at a temperature from about 0° C. to about 50° C., preferably about ambient temperature. Alternatively, the protecting groups are conveniently attached using Schotten-Baumann conditions.

Subsequent to the above described coupling reaction, of compounds 2 and 5, the protecting group may be removed by deprotecting methods known to those skilled in the art such as treatment with trifluoroacetic acid in methylene chloride for the tert-butoxycarbonyl protected products.

For a description of protecting groups and their use, see T. W. Greene and P. G. M. Wuts, “Protective Groups in Organic Synthesis” Second Ed., John Wiley & Sons, New York, 1991.

For the production of compounds of Formula I wherein R¹ or R² is hydroxy, cleavage of a Formula I compound wherein R¹ or R² is (C₁-C₄)alkoxy is preferred.

The cleavage reaction may conveniently be carried out by any of the many procedures known for such a transformation. Treatment of the protected formula I derivative with molten pyridine hydrochloride (20-30 eq.) at 150° to 175° C. may be employed for O-dealkylations. Alternatively, the cleavage reaction may be carried out, for example, by treatment of the protected quinazoline derivative with an alkali metal (C₁-C₄)alkylsulphide, such as sodium ethanethiolate or by treatment with an alkali metal diarylphosphide such as lithium diphenylphosphide. The cleavage reaction may also, conveniently, be carried out by treatment of the protected quinazoline derivative with a boron or aluminum trihalide such as boron tribromide. Such reactions are preferably carried out in the presence of a reaction-inert solvent at a suitable temperature.

Compounds of formula I, wherein R¹ or R² is a (C₁-C₄)alkylsulphinyl or (C₁-C₄)alkylsulphonyl group are preferably prepared by oxidation of a formula I compound wherein R¹ or R² is a (C₁-C₄)alkylsulfanyl group. Suitable oxidizing agents are known in the art for the oxidation of sulfanyl to sulphinyl and/or sulphonyl, e.g., hydrogen peroxide, a peracid (such as 3-chloroperoxybenzoic or peroxyacetic acid), an alkali metal peroxysulphate (such as potassium peroxymonosulphate), chromium trioxide or gaseous oxygen in the presence of platinum. The oxidation is generally carried out under as mild conditions as possible using the stoichiometric amount of oxidizing agent in order to reduce the risk of over oxidation and damage to other functional groups. In general, the reaction is carried out in a suitable solvent such as methylene chloride, chloroform, acetone, tetrahydrofuran or tert-butyl methyl ether and at a temperature from about −25° to 50° C., preferably at or near ambient temperature, i.e., in the range of 15° to 35° C. When a compound carrying a sulphinyl group is desired a milder oxidizing agents should be used such as sodium or potassium metaperiodate, conveniently in a polar solvent such as acetic acid or ethanol. The compounds of formula I containing a (C₁-C₄)alkylsulphonyl group may be obtained by oxidation of the corresponding (C₁-C₄)alkylsulphinyl compound as well as of the corresponding (C₁-C₄)alkylsulfanyl compound.

Compounds of formula I wherein R¹ is optionally substituted (C₂-C₄)alkanoylamino, ureido, 3-phenylureido, benzamido or sulfonamido can be prepared by acylation or sulfonylation of a corresponding compound wherein R¹ is amino. Suitable acylating agents are any agents known in the art for the acylation of amino to acylamino, for example, acyl halides, e.g., a (C₂-C₄)alkanoyl chloride or bromide or a benzoyl chloride or bromide, alkanoic acid anhydrides or mixed anhydrides (e.g., acetic anhydride or the mixed anhydride formed by the reaction of an alkanoic acid and a (C₁-C₄) alkoxycarbonyl halide, for example (C₁-C₄)alkoxycarbonyl chloride, in the presence of a suitable base. For the production of those compounds of Formula I wherein R¹ is ureido or 3-phenylureido, a suitable acylating agent is, for example, a cyanate, e.g., an alkali metal cyanate such as sodium cyanate, or an isocyanate such as phenyl isocyanate. N-sulfonylations may be carried out with suitable sulfonyl halides or sulfonylanhydrides in the presence of a tertiary amine base. In general the acylation or sulfonylation is carried out in a reaction-inert solvent and at a temperature in the range of about −30° to 120° C., conveniently at or near ambient temperature.

Compounds of Formula I wherein R¹ is (C₁-C₄)alkoxy or substituted (C₁-C₄)alkoxy or R¹ is (C₁-C₄)alkylamino or substituted mono-N— or di-N,N—(C₁-C₄)alkylamino, are prepared by the alkylation, preferably in the presence of a suitable base, of a corresponding compound wherein R¹ is hydroxy or amino, respectively. Suitable alkylating agents include alkyl or substituted alkyl halides, for example, an optionally substituted (C₁-C₄)alkyl chloride, bromide or iodide, in the presence of a suitable base in a reaction-inert solvent and at a temperature in the range of about 10° to 140° C., conveniently at or near ambient temperature.

For the production of those compounds of Formula I wherein R¹ is an amino-, oxy- or cyano-substituted (C₁-C₄)alkyl substituent, a corresponding compound wherein R¹ is a (C₁-C₄)alkyl substituent bearing a group which is displacable by an amino-, alkoxy-, or cyano group is reacted with an appropriate amine, alcohol or cyanide, preferably in the presence of a suitable base. The reaction is preferably carried out in a reaction-inert solvent or diluent and at a temperature in the range of about 10° to 100° C., preferably at or near ambient temperature.

Compounds of Formula I, wherein R¹ is a carboxy substituent or a substituent which includes a carboxy group are prepared by hydrolysis of a corresponding compound wherein R¹ is a (C₁-C₄)alkoxycarbonyl substituent or a substituent which includes a (C₁-C₄)alkoxycarbonyl group. The hydrolysis may conveniently be performed, for example, under basic conditions, e.g., in the presence of alkali metal hydroxide.

Compounds of Formula I wherein R¹ is amino, (C₁-C₄)alkylamino, di-[(C₁-C₄)alkyllamino, pyrrolidin-1-yl, piperidino, morpholino, piperazin-1-yl, 4-(C₁-C₄)alkylpiperazin-1-yl or (C₁-C₄)alkysulfanyl, may be prepared by the reaction, in the presence of a suitable base, of a corresponding compound wherein R¹ is an amine or thiol displaceable group with an appropriate amine or thiol. The reaction is preferably carried out in a reaction-inert solvent or diluent and at a temperature in the range of about 10° to 180° C., conveniently in the range 100° to 150° C.

Compounds of Formula I wherein R¹ is 2-oxopyrrolidin-1-yl or 2-oxopiperidin-1-yl are prepared by the cyclisation, in the presence of a suitable base, of a corresponding compound wherein RI is a halo-(C₂-C₄)alkanoylamino group. The reaction is preferably carried out in a reaction-inert solvent or diluent and at a temperature in the range of about 10° to 100° C., conveniently at or near ambient temperature.

For the production of compounds of Formula I in which R¹ is carbamoyl, substituted carbamoyl, alkanoyloxy or substituted alkanoyloxy, the carbamoylation or acylation of a corresponding compound wherein R¹ is hydroxy is convenient.

Suitable acylating agents known in the art for acylation of hydroxyaryl moieties to alkanoyloxyaryl groups include, for example, (C₂-C₄)alkanoyl halides, (C₂-C₄)alkanoyl anhydrides and mixed anhydrides as described above, and suitable substituted derivatives thereof may be employed, typically in the presence of a suitable base. Alternatively, (C₂-C₄)alkanoic acids or suitably substituted derivatives thereof may be coupled with a Formula I compound wherein R¹ is hydroxy with the aid of a condensing agent such as a carbodiimide. For the production of those compounds of Formula I in which R¹ is carbamoyl or substituted carbamoyl, suitable carbamoylating agents are, for example, cyanates or alkyl or arylisocyanates, typically in the presence of a suitable base. Alternatively, suitable intermediates such as the chloroformate or carbonylimidazolyl derivative of a compound of Formula I in which R¹ is hydroxy may be generated, for example, by treatment of said derivative with phosgene (or a phosgene equivalent) or carbonyidiimidazole. The resulting intermediate may then be reacted with an appropriate amine or substituted amine to produce the desired carbamoyl derivatives.

Compounds of formula I wherein R¹ is aminocarbonyl or a substituted aminocarbonyl can be prepared by the aminolysis of a suitable intermediate in which R¹ is carboxy.

The activation and coupling of formula I compounds wherein R¹ is carboxy may be performed by a variety of methods known to those skilled in the art. Suitable methods include activation of the carboxyl as an acid halide, azide, symmetric or mixed anhydride, or active ester of appropriate reactivity for coupling with the desired amine. Examples of such types of intermediates and their production and use in couplings with amines may be found extensively in the literature; for example M. Bodansky and A. Bodansky, “The Practice of Peptide Synthesis”, Springer-Verlag, N.Y., 1984. The resulting formula I compounds may be isolated and purified by standard methods, such as solvent removal and recrystallization or chromatography.

The starting materials for the described reaction scheme I (e.g., amines, quinazolines and amine protecting groups) are readily available or can be easily synthesized by those skilled in the art using conventional methods of organic synthesis. For example, the preparation of 2,3-dihydro-1,4-benzoxazine derivatives are described in R. C. Elderfield, W. H. Todd, S. Gerber, Ch. 12 in “Heterocyclic Compounds”, Vol. 6, R. C. Elderfield ed., John Wiley and Sons, Inc., N.Y., 1957. Substituted 2,3-dihydrobenzothiazinyl compounds are described by R. C. Elderfield and E. E. Harris in Ch. 13 of Volume 6 of the Elderfield “Heterocyclic Compounds” book.

In another particular embodiment, the EGFR inhibitor has a general formula II as described in U.S. Pat. No. 5,457,105: 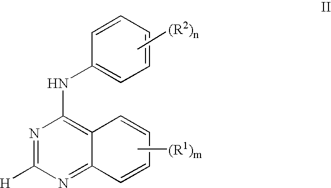
wherein:

m is 1, 2 or 3 and

each R¹ is independently 6-hydroxy, 7-hydroxy, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkoxyamino, (2-4C)alkanoyloxyamino, trifluoromethoxy, (1-4C)alkyl, 6-(1-4C)alkoxy, 7-(1-4C)alkoxy, (1-3C)alkylenedioxy, (1-4C)alkylamino, di-1[(1-4C)alkyl)amino, pyrrolidin-1-yl, piperidino, morpholino, piperazin-1-yl, 4-(1-4C)alkylpiperazin-1-yl, (1-4C)alkylthio, (1-4C)alkylsulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, carboxy-(1-4C)alkyl, (1-4C)alkoxycarbonyl-(1-4C)alkyl, carbamoyl-(1-4C)alkyl, N-(1-4C)alkylcarbamoyl-(1-4C)alkyl, N,N-di-[(1-4C)alkyl]carbamoyl-(1-4C)alkyl, amino-(1-4C)alkyl, (1- 4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C) alkyl, 4-(1-4C)alkylpiperazin-1-yl-(1-4C) alkyl, hydroxy-(2-4C)alkoxy-(1-4C) alkyl, (1-4C)alkoxy-(2-4C)alkoxy-(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, phenoxy-(1-4C)alkyl, anilino-(1-4C)alkyl, phenylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C) alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, (2-4C)alkanoyloxy, hydroxy-(2-4C)alkanoyloxy, (1-4C)alkoxy-(2-4C)alkanoyloxy, phenyl-(1-4C)alkoxy, phenoxy-(2-4C)alkoxy, anilino-(2-4C)alkoxy, phenylthio-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, carboxy-(1-4C)alkylamino, (1-4C)alkoxycarbonyl-(1-4C)alkylamino, carbamoyl-(1-4C)alkylamino, N-(1-4C)alkylcarbamoyl-(1-4C)alkylamino, N,N-di-[(1-4C)alkyllcarbamoyl-(1-4C)alkylamino, amino-(2-4C)alkylamino, (1-4C)alkylamino-(2-4C)alkylamino, di-1(1-4C)alkyllamino-(2-4C)alkylamino, phenyl-(1-4C)alkylamino, phenoxy-(2-4C)alkylamino, anilino-(2-4C)alkylamino, phenylthio-(2-4C)alkylamino, (2-4C)alkanoylamino, (1-4C)alkoxycarbonylamino, (1-4C)alkylsulphonylamino, benzamido, benzenesulphonamido, 3-phenylureido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, carbamoyl-(2-4C)alkanoylamino, N-(1-4C)alkylcarbamoyl-(2-4C)alkanoylamino, N,N-di-[(1-4C)alkyllcarbamoyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent or any anilino, phenoxy or phenyl group in a R¹ substituent may optionally bear one or two halogeno, (1-4C)alkyl or (1-4C)alkoxy substituents;

n is 1 or 2 and

each R² is independently hydrogen, hydroxy, halogeno, trifluoromethyl, amino, nitro, cyano, (1-4C)alkyl, (1-4C)alkoxy, (1-4C)alkylamino, di-[(1-4C)alkyl]amino, (1-4C)alkylthio, (1-4C)alkylsulphinyl or (1-4C)alkylsulphonyl; or a pharmaceutically-acceptable salt thereof; except that 4-(4′- hydroxyanilino)-6-methoxyquinazoline, 4-(4,-hydroxyanilino)-6,7-methylenedioxyquinazoline, 6-amino-4-(4′-aminoanilino)quinazoline, 4-anilino-6-methylquinazoline or the hydrochloride salt thereof and 4-anilino-6,7-dimethoxyquinazoline or the hydrochloride salt thereof are excluded.

In a particular embodiment, the EGFR inhibitor is a compound according to formula II selected from the group consisting of:
4-(3′-chloro-4′-fluoroanilino)-6,7-dimethoxyquinazoline; 4-(3′,4′-dichloroanilino)-6,7-dimethoxyquinazoline; 6,7-dimethoxy-4-(3′-nitroanilino)-quinazoline; 6,7-diethoxy-4-(3′-methylanilino)-quinazoline; 6-methoxy-4-(3′-methylanilino)-quinazoline; 4-(3′-chloroanilino)-6-methoxyquinazoline; 6,7-ethylenedioxy-4-(3′-methylanilino)-quinazoline; 6-amino-7-methoxy-4-(3′-methylanilino)-quinazoline; 4-(3′-methylanilino)-6-ureidoquinazoline; 6-(2-methoxyethoxymethyl)-4-(3′-methylanilino)-quinazoline; 6,7-di-(2-methoxyethoxy)-4-(3′-methylanilino)-quinazoline; 6-dimethylamino-4-(3′-methylanilino)quinazoline; 6-benzamido-4-(3′-methylanilino)quinazoline; 6,7-dimethoxy-4-(3′-trifluoromethylanilino)-quinazoline; 6-hydroxy-7-methoxy-4-(3′-methylanilino)-quinazoline; 7-hydroxy-6-methoxy-4-(3′-methylanilino)-quinazoline; 7-amino-4-(3′-methylanilino)-quinazoline; 6-amino-4-(3′-methylanilino)quinazoline; 6-amino-4-(3′-chloroanilino)-quinazoline; 6-acetamido-4-(3′-methylanilino)-quinazoline; 6-(2-methoxyethylamino)-4-(3′-methylanilino)-quinazoline; 7-(2- methoxyacetamido)-4-(3′-methylanilino)-quinazoline; 7-(2-hydroxyethoxy)-6-methoxy-4-(3′-methylanilino)-quinazoline; 7-(2-methoxyethoxy)-6-methoxy-4-(3′-methylanilino)-quinazoline; 6-amino-4-(3′-methylanilino)-quinazoline.

A quinazoline derivative of the formula II, or a pharmaceutically-acceptable salt thereof, may be prepared by any process known to be applicable to the preparation of chemically-related compounds. A suitable process is, for example, illustrated by that used in U.S. Pat. No. 4,322,420. Necessary starting materials may be commercially available or obtained by standard procedures of organic chemistry.

(a) The reaction, conveniently in the presence of a suitable base, of a quinazoline (i), wherein Z is a displaceable group, with an aniline (ii).

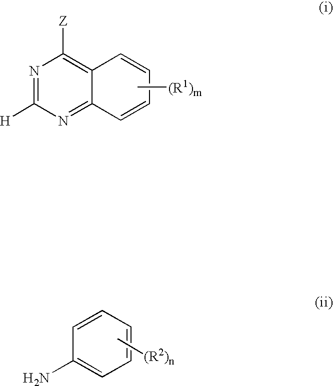

A suitable displaceable group Z is, for example, a halogeno, alkoxy, aryloxy or sulphonyloxy group, for example a chloro, bromo, methoxy, phenoxy, methanesulphonyloxy or toluene-p-sulphonyloxy group.

A suitable base is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or for example, an alkali or alkaline earth metal carbonate or hydroxide, for example sodium carbonate, potassium carbonate, calcium carbonate, sodium hydroxide or potassium hydroxide.

The reaction is preferably carried out in the presence of a suitable inert solvent or diluent, for example an alkanol or ester such as methanol, ethanol, isopropanol or ethyl acetate, a halogenated solvent such as methylene chloride, chloroform or carbon tetrachloride, an ether such as tetrahydrofuran or 1,4-dioxan, an aromatic solvent such as toluene, or a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 10° to 150° C., preferably in the range 20° to 80° C.

The quinazoline derivative of the formula II may be obtained from this process in the form of the free base or alternatively it may be obtained in the form of a salt with the acid of the formula H-Z wherein Z has the meaning defined hereinbefore. When it is desired to obtain the free base from the salt, the salt may be treated with a suitable base as defined hereinbefore using a conventional procedure.

(b) For the production of those compounds of the formula II wherein R¹ or R² is hydroxy, the cleavage of a quinazoline derivative of the formula II wherein R¹ or R² is (1-4C)alkoxy.

The cleavage reaction may conveniently be carried out by any of the many procedures known for such a transformation. The reaction may be carried out, for example, by treatment of the quinazoline derivative with an alkali metal (1-4C)alkylsulphide such as sodium ethanethiolate or, for example, by treatment with an alkali metal diarylphosphide such as lithium diphenylphosphide. Alternatively the cleavage reaction may conveniently be carried out, for example, by treatment of the quinazoline derivative with a boron or aluminium trihalide such as boron tribromide. Such reactions are preferably carried out in the presence of a suitable inert solvent or diluent as defined hereinbefore and at a suitable temperature.

(c) For the production of those compounds of the formula II wherein R¹ or R² is a (1-4C)alkylsulphinyl or (1-4C)alkylsulphonyl group, the oxidation of a quinazoline derivative of the formula II wherein R¹ or R² is a (1-4C)alkylthio group.

A suitable oxidising agent is, for example, any agent known in the art for the oxidation of thio to sulphinyl and/or sulphonyl, for example, hydrogen peroxide, a peracid (such as 3-chloroperoxybenzoic or peroxyacetic acid), an alkali metal peroxysulphate (such as potassium peroxymonosulphate), chromium trioxide or gaseous oxygen in the presence of platinium. The oxidation is generally carrried out under as mild conditions as possible and with the required stoichiometric amount of oxidising agent in order to reduce the risk of over oxidation and damage to other functional groups. In general the reaction is carried out in a suitable solvent or diluent such as methylene chloride, chloroform, acetone, tetrahydrofuran or tert-butyl methyl ether and at a temperature, for example, −25° to 50° C., conveniently at or near ambient temperature, that is in the range 15° to 35° C. When a compound carrying a sulphinyl group is required a milder oxidising agent may also be used, for example sodium or potassium metaperiodate, conveniently in a polar solvent such as acetic acid or ethanol. It will be appreciated that when a compound of the formula II containing a (1-4C)alkylsulphonyl group is required, it may be obtained by oxidation of the corresponding (1-4C)alkylsulphinyl compound as well as of the corresponding (1-4C)alkylthio compound.

(d) For the production of those compounds of the formula II wherein R¹ is amino, the reduction of a quinazoline derivative of the formula I wherein R¹ is nitro.

The reduction may conveniently be carried out by any of the many procedures known for such a transformation. The reduction may be carrried out, for example, by the hydrogenation of a solution of the nitro compound in an inert solvent or diluent as defined hereinbefore in the presence of a suitable metal catalyst such as palladium or platinum. A further suitable reducing agent is, for example, an activated metal such as activated iron (produced by washing iron powder with a dilute solution of an acid such as hydrochloric acid). Thus, for example, the reduction may be carried out by heating a mixture of the nitro compound and the activated metal in a suitable solvent or diluent such as a mixture of water and an alcohol, for example, methanol or ethanol, to a temperature in the range, for example, 50° to 150° C., conveniently at or near 70° C.

(e) For the production of those compounds of the formula II wherein R¹ is (2-4C)alkanoylamino or substituted (2-4C)alkanoylamino, ureido, 3-phenylureido or benzamido, or R² is acetamido or benzamido, the acylation of a quinazoline derivative of the formula II wherein R¹ or R² is amino.

A suitable acylating agent is, for example, any agent known in the art for the acylation of amino to acylamino, for example an acyl halide, for example a (2-4C)alkanoyl chloride or bromide or a benzoyl chloride or bromide, conveniently in the presence of a suitable base, as defined hereinbefore, an alkanoic acid anhydride or mixed anhydride, for example a (2-4C)alkanoic acid anhydride such as acetic anhydride or the mixed anhydride formed by the reaction of an alkanoic acid and a (1-4C)alkoxycarbonyl halide, for example a (1-4C)alkoxycarbonyl chloride, in the presence of a suitable base as defined hereinbefore. For the production of those compounds of the formula II wherein R¹ is ureido or 3-phenylureido, a suitable acylating agent is, for example, a cyanate, for example an alkali metal cyanate such as sodium cyanate or, for example, an isocyanate such as phenyl isocyanate. In general the acylation is carried out in a suitable inert solvent or diluent as defined hereinbefore and at a temperature, in the range, for example, −30° to 120° C., conveniently at or near ambient temperature.

(f) For the production of those compounds of the formula II wherein R¹ is (1-4C)alkoxy or substituted (1-4C)alkoxy or R¹ is (1-4C)alkylamino or substituted (1-4C)alkylamino, the alkylation, preferably in the presence of a suitable base as defined hereinbefore, of a quinazoline derivative of the formula II wherein R¹ is hydroxy or amino as appropriate.

A suitable alkylating agent is, for example, any agent known in the art for the alkylation of hydroxy to alkoxy or substituted alkoxy, or for the alkylation of amino to alkylamino or substituted alkylamino, for example an alkyl or substituted alkyl halide, for example a (1-4C)alkyl chloride, bromide or iodide or a substituted (1-4C)alkyl chloride, bromide or iodide, in the presence of a suitable base as defined hereinbefore, in a suitable inert solvent or diluent as defined hereinbefore and at a temperature in the range, for example, 10° to 140° C., conveniently at or near ambient temperature.

(g) For the production of those compounds of the formula II wherein R¹ is a carboxy substituent or a substituent which includes a carboxy group, the hydrolysis of a quinazoline derivative of the formula II wherein R¹ is a (1-4C)alkoxycarbonyl substituent or a substituent which includes a (1-4C)alkoxycarbonyl group.

The hydrolysis may conveniently be performed, for example, under basic conditions.

(h) For the production of those compounds of the formula II wherein R¹ is an amino-, oxy-, thio- or cyano-substituted (1-4C)alkyl substituent, the reaction, preferably in the presence of a suitable base as defined hereinbefore, of a quinazoline derivative of the formula II wherein R¹ is a (1-4C)alkyl substituent bearing a displaceable group as defined hereinbefore with an appropriate amine, alcohol, thiol or cyanide.

The reaction is preferably carried out in a suitable inert solvent or diluent as defined hereinbefore and at a temperature in the range, for example, 10° to 100° C., conveniently at or near ambient temperature.

When a pharmaceutically-acceptable salt of a quinazoline derivative of the formula II is required, it may be obtained, for example, by reaction of said compound with, for example, a suitable acid using a conventional procedure.

In a particular embodiment, the EGFR inhibitor is a compound according to formula II′ as disclosed in U.S. Pat. No. 5,770,599:

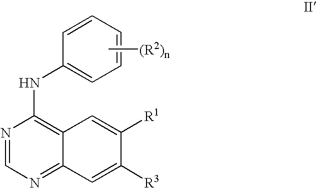

wherein n is 1, 2 or 3;

each R² is independently halogeno or trifluoromethyl

R³ is (1-4C)alkoxy; and

R¹ is di-[(1-4C)alkyl]amino-(2-4C)alkoxy, pyrrolidin-1-yl-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, imidazol-1-yl-(2-4C)alkoxy, di-[(1-4C)alkoxy-(2-4C)alkyllamino-(2-4C)alkoxy, thiamorpholino-(2-4C)alkoxy, 1-oxothiamorpholino-(2-4C)alkoxy or 1,1-dioxothiamorpholino-(2-4C)alkoxy, and wherein any of the above mentioned R¹ substituents comprising a CH₂ (methylene) group which is not attached to a N or O atom optionally bears on said CH₂ group a hydroxy substituent;

or a pharmaceutically-acceptable salt thereof.

In a particular embodiment, the EGFR inhibitor is a compound according to formula II′ selected from the group consisting of:
4-(3′-chloro-4′-fluoroanilino)-7-methoxy-6-(2-pyrrolidin-1-ylethoxy)-quinazoline; 4-(3′-chloro-4′-fluoroanilino)-7-methoxy-6-(2-morpholinoethoxy)-quinazoline; 4-(3′-chloro-4′-fluoroanilino)-6-(3-diethylaminopropoxy)-7- methoxyquinazoline; 4-(3′-chloro-4′-fluoroanilino)-7-methoxy-6-(3-pyrrolidin-l-ylpropoxy)-quinazoline; 4-(3′-chloro-4′-fluoroanilino)-6-(3-dimethylaminopropoxy)-7-methoxyquinazoline; 4-(3′,4′-difluoroanilino)-7-methoxy-6-(3-morpholinopropoxy)-quinazoline; 4-(3′-chloro-4′-fluoroanilino)-7-methoxy-6-(3-piperidinopropoxy)-quinazoline; 4-(3′-chloro-4′-fluoroanilino)-7-methoxy-6-(3-morpholinopropoxy)-quinazoline; 4-(3′-chloro-4′-fluoroanilino)-6-(2-dimethylaminoethoxy)-7-methoxyquinazoline; 4-(2′,4′-difluoroanilino)-6-(3-dimethylaminopropoxy)-7-methoxyquinazoline; 4-(2′,4′-difluoroanilino)-7-methoxy-6-(3-morpholinopropoxy)-quinazoline; 4-(3′-chloro-4′-fluoroanilino)-6-(2-imidazol-1-ylethoxy)-7-methoxyquinazoline; 4-(3′-chloro-4′-fluoroanilino)-6-(3-imidazol-1-ylpropoxy)-7-methoxyquinazoline; 4-(3′-chloro-4′-fluoroanilino)-6-(2-dimethylaminoethoxy)-7-methoxyquinazoline; 4-(2′,4′-difluoroanilino)-6-(3-dimethylaminopropoxy)-7-methoxyquinazoline; 4-(2′,4′-difluoroanilino)-7-methoxy-6-(3-morpholinopropoxy)-quinazoline; 4-(3′-chloro-4′-fluoroanilino)-6-(2-imidazol-1-ylethoxy)-7-methoxyquinazoline; and 4-(3′-chloro-4′-fluoroanilino)-6-(3-imidazol-1-ylpropoxy)-7-methoxyquinazoline.

In a particular embodiment, the EGFR inhibitor is a compound according to formula II′ that is 4-(3′-chloro-4′-fluoroanilino)-7-methoxy-6-(3-morpholinopropoxy)-quinazoline, alternatively referred to as ZD 1839, gefitinib and Iressa™.

A quinazoline derivative of the formula II′, or a pharmaceutically-acceptable salt thereof, may be prepared by any process known to be applicable to the preparation of chemically-related compounds. Suitable processes include, for example, those illustrated in U.S. Pat. Nos. 5,616,582, 5,580,870, 5,475,001 and 5,569,658. Unless otherwise stated, n, R 2, R³ and R¹ have any of the meanings defined hereinbefore for a quinazoline derivative of the formula II′. Necessary starting materials may be commercially available or obtained by standard procedures of organic chemistry.

(a) The reaction, conveniently in the presence of a suitable base, of a quinazoline (iii) wherein Z is a displaceable group, with an aniline (iv)

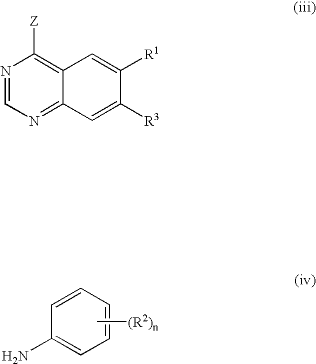

A suitable displaceable group Z is, for example, a halogeno, alkoxy, aryloxy or sulphonyloxy group, for example a chloro, bromo, methoxy, phenoxy, methanesulphonyloxy or toluene-4-sulphonyloxy group.

A suitable base is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or for example, an alkali or alkaline earth metal carbonate or hydroxide, for example sodium carbonate, potassium carbonate, calcium carbonate, sodium hydroxide or potassium hydroxide. Alternatively a suitable base is, for example, an alkali metal or alkaline earth metal amide, for example sodium amide or sodium bis(trimethylsilyl)amide.

The reaction is preferably carried out in the presence of a suitable inert solvent or diluent, for example an alkanol or ester such as methanol, ethanol, isopropanol or ethyl acetate, a halogenated solvent such as methylene chloride, chloroform or carbon tetrachloride, an ether such as tetrahydrofuran or 1,4-dioxan, an aromatic solvent such as toluene, or a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 10° to 150° C., preferably in the range 20° to 80° C.

The quinazoline derivative of the formula II′ may be obtained from this process in the form of the free base or alternatively it may be obtained in the form of a salt with the acid of the formula H-Z wherein Z has the meaning defined hereinbefore. When it is desired to obtain the free base from the salt, the salt may be treated with a suitable base as defined hereinbefore using a conventional procedure.

(b) For the production of those compounds of the formula II′ wherein R¹ is an amino-substituted (2-4C)alkoxy group, the alkylation, conveniently in the presence of a suitable base as defined hereinbefore, of a quinazoline derivative of the formula II′ wherein R¹ is a hydroxy group.

A suitable alkylating agent is, for example, any agent known in the art for the alkylation of hydroxy to amino-substituted alkoxy, for example an amino-substituted alkyl halide, for example an amino-substituted (2-4C)alkyl chloride, bromide or iodide, in the presence of a suitable base as defined hereinbefore, in a suitable inert solvent or diluent as defined hereinbefore and at a temperature in the range, for example, 10° to 140° C., conveniently at or near 80° C.

(c) For the production of those compounds of the formula II′ wherein R¹ is an amino-substituted (2-4C)alkoxy group, the reaction, conveniently in the presence of a suitable base as defined hereinbefore, of a compound of the formula II′ wherein R¹ is a hydroxy-(2-4C)alkoxy group, or a reactive derivative thereof, with an appropriate amine.

A suitable reactive derivative of a compound of the formula II′ wherein R¹ is a hydroxy-(2-4C)alkoxy group is, for example, a halogeno- or sulphonyloxy-(2-4C)alkoxy group such as a bromo- or methanesulphonyloxy-(2-4C)alkoxy group.

The reaction is preferably carried out in the presence of a suitable inert solvent or diluent as defined hereinbefore and at a temperature in the range, for example, 10° to 150° C., conveniently at or near 50° C.

(d) For the production of those compounds of the formula II′ wherein R¹ is a hydroxy-amino-(2-4C)alkoxy group, the reaction of a compound of the formula II′ wherein R¹ is a 2,3-epoxypropoxy or 3,4-epoxybutoxy group with an appropriate amine.

The reaction is preferably carried out in the presence of a suitable inert solvent or diluent as defined hereinbefore and at a temperature in the range, for example, 10° to 150° C., conveniently at or near 70° C.

When a pharmaceutically-acceptable salt of a quinazoline derivative of the formula II′ is required, for example a mono- or di-acid-addition salt of a quinazoline derivative of the formula II′, it may be obtained, for example, by reaction of said compound with, for example, a suitable acid using a conventional procedure.

### II. Compositions and Methods of the Invention

A cytokine related to the TNF ligand family, the cytokine identified herein as “Apo-2 ligand” or “TRAIL” has been described. The predicted mature amino acid sequence of native human Apo-2 ligand contains 281 amino acids, and has a calculated molecular weight of approximately 32.5 kDa. The absence of a signal sequence and the presence of an internal hydrophobic region suggest that Apo-2 ligand is a type II transmembrane protein. Soluble extracellular domain Apo-2 ligand polypeptides have also been described. See, e.g., WO97/25428 published Jul. 17, 1997. Apo-2L substitutional variants have further been described. Alanine scanning techniques have been utilized to identify various substitutional variant molecules having biological activity. Particular substitutional variants of the Apo-2 ligand include those in which at least one amino acid is substituted by another amino acids such as an alanine residue. These substitutional variants are identified, for example, as “D203A”; “D218A” and “D269A.” This nomenclature is used to identify Apo-2 ligand variants wherein the aspartic acid residues at positions 203, 218, and/or 269 (using the numbering shown in FIG. 1) are substituted by alanine residues. Optionally, the Apo-2L variants of the present invention may comprise one or more of the amino acid substitutions. Optionally, such Apo-2L variants will be DR4 or DR5 receptor selective variants.

The description below relates to methods of producing Apo-2 ligand, including Apo-2 ligand variants, by culturing host cells transformed or transfected with a vector containing Apo-2 ligand encoding nucleic acid and recovering the polypeptide from the cell culture.

The DNA encoding Apo-2 ligand may be obtained from any cDNA library prepared from tissue believed to possess the Apo-2 ligand MRNA and to express it at a detectable level. Accordingly, human Apo-2 ligand DNA can be conveniently obtained from a cDNA library prepared from human tissues, such as the bacteriophage library of human placental cDNA as described in WO97/25428. The Apo-2 ligand-encoding gene may also be obtained from a genomic library or by oligonucleotide synthesis.

Libraries can be screened with probes (such as antibodies to the Apo-2 ligand or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding Apo-2 ligand is to use PCR methodology (Sambrook et al., supra; Dieffenbach et al., PCR Primer:A Laboratory Manual (Cold Spring Harbor Laboratory Press, 1995)].

Amino acid sequence fragments or variants of Apo-2 ligand can be prepared by introducing appropriate nucleotide changes into the Apo-2 ligand DNA, or by synthesis of the desired Apo-2 ligand polypeptide. Such fragments or variants represent insertions, substitutions, and/or deletions of residues within or at one or both of the ends of the intracellular region, the transmembrane region, or the extracellular region, or of the amino acid sequence shown for the full-length Apo-2 ligand in FIG. 1. Any combination of insertion, substitution, and/or deletion can be made to arrive at the final construct, provided that the final construct possesses, for instance, a desired biological activity, such as apoptotic activity, as defined herein. In a preferred embodiment, the fragments or variants have at least about 80% amino acid sequence identity, more preferably, at least about 90% sequence identity, and even more preferably, at least 95%, 96%, 97%, 98% or 99% sequence identity with the sequences identified herein for the intracellular, transmembrane, or extracellular domains of Apo-2 ligand, or the full-length sequence for Apo-2 ligand. The amino acid changes also may alter post-translational processes of the Apo-2 ligand, such as changing the number or position of glycosylation sites or altering the membrane anchoring characteristics.

Variations in the Apo-2 ligand sequence as described above can be made using any of the techniques and guidelines for conservative and non-conservative mutations set forth in U.S. Pat. No. 5,364,934. These include oligonucleotide-mediated (site-directed) mutagenesis, alanine scanning, and PCR mutagenesis.

Scanning amino acid analysis can be employed to identify one or more amino acids along a contiguous sequence. Among the preferred scanning amino acids are relatively small, neutral amino acids. Such amino acids include alanine, glycine, serine and cysteine. Alanine is typically a preferred scanning amino acid among this group because it eliminates the side-chain beyond the beta-carbon and is less likely to alter the main-chain conformation of the variant. (Cunningham et al., Science, 244:1081 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)].

Amino acids may be grouped according to similarities in the properties of their side chains (in A. L. Lehninger, in Biochemistry, second ed., pp. 73-75, Worth Publishers, New York (1975)):
(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Variations in the Apo-2 ligand sequence also included within the scope of the invention relate to amino-terminal derivatives or modified forms. Such Apo-2 ligand sequences include any of the Apo-2 ligand polypeptides described herein having a methionine or modified methionine (such as formyl methionyl or other blocked methionyl species) at the N-terminus of the polypeptide sequence.

The nucleic acid (e.g., cDNA or genomic DNA) encoding native or variant Apo-2 ligand may be inserted into a replicable vector for further cloning (amplification of the DNA) or for expression. Various vectors are publicly available. The vector components generally include, but are not limited to, one or more of the following: a signal sequence, an origin of replication, one or more marker genes, an enhancer element, a promoter, and a transcription termination sequence, each of which is described below. Optional signal sequences, origins of replication, marker genes, enhancer elements and transcription terminator sequences that may be employed are known in the art and described in further detail in WO97/25428.

Expression and cloning vectors usually contain a promoter that is recognized by the host organism and is operably linked to the Apo-2 ligand nucleic acid sequence. Promoters are untranslated sequences located upstream (5′) to the start codon of a structural gene (generally within about 100 to 1000 bp) that control the transcription and translation of a particular nucleic acid sequence, such as the Apo-2 ligand nucleic acid sequence, to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to Apo-2 ligand encoding DNA by removing the promoter from the source DNA by restriction enzyme digestion and inserting the isolated promoter sequence into the vector. Both the native Apo-2 ligand promoter sequence and many heterologous promoters may be used to direct amplification and/or expression of the Apo-2 ligand DNA.

Promoters suitable for use with prokaryotic and eukaryotic hosts are known in the art, and are described in further detail in WO97/25428.

A preferred method for the production of soluble Apo-2L in E. coli employs an inducible promoter for the regulation of product expression. The use of a controllable, inducible promoter allows for culture growth to the desirable cell density before induction of product expression and accumulation of significant amounts of product which may not be well tolerated by the host.

Several inducible promoter systems (T7 polymerase, trp and alkaline phosphatase (AP)) have been evaluated by Applicants for the expression of Apo-2L (form 114-281). The use of each of these three promoters resulted in significant amounts of soluble, biologically active Apo-2L trimer being recovered from the harvested cell paste. The AP promoter is preferred among these three inducible promoter systems tested because of tighter promoter control and the higher cell density and titers reached in harvested cell paste.

Construction of suitable vectors containing one or more of the above-listed components employs standard ligation techniques. Isolated plasmids or DNA fragments are cleaved, tailored, and re-ligated in the form desired to generate the plasmids required.

For analysis to confirm correct sequences in plasmids constructed, the ligation mixtures can be used to transform E. coli K12 strain 294 (ATCC 31,446) and successful transformants selected by ampicillin or tetracycline resistance where appropriate. Plasmids from the transformants are prepared, analyzed by restriction endonuclease digestion, and/or sequenced using standard techniques known in the art. [See, e.g., Messing et al., Nucleic Acids Res., 9:309 (1981); Maxam et al., Methods in Enzymology, 65:499 (1980)].

Expression vectors that provide for the transient expression in mammalian cells of DNA encoding Apo-2 ligand may be employed. In general, transient expression involves the use of an expression vector that is able to replicate efficiently in a host cell, such that the host cell accumulates many copies of the expression vector and, in turn, synthesizes high levels of a desired polypeptide encoded by the expression vector [Sambrook et al., supral. Transient expression systems, comprising a suitable expression vector and a host cell, allow for the convenient positive identification of polypeptides encoded by cloned DNAs, as well as for the rapid screening of such polypeptides for desired biological or physiological properties. Thus, transient expression systems are particularly useful in the invention for purposes of identifying analogs and variants of Apo-2 ligand that are biologically active Apo-2 ligand.

Other methods, vectors, and host cells suitable for adaptation to the synthesis of Apo-2 ligand in recombinant vertebrate cell culture are described in Gething et al., Nature, 293:620-625 (1981); Mantei et al., Nature, 281:40-46 (1979); EP 117,060; and EP 117,058.

Suitable host cells for cloning or expressing the DNA in the vectors herein include prokaryote, yeast, or higher eukaryote cells. Suitable prokaryotes for this purpose include but are not limited to eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. Preferably, the host cell should secrete minimal amounts of proteolytic enzymes.

In addition to prokaryotes, eukaryotic microbes such as filamentous fungi or yeast are suitable cloning or expression hosts for Apo-2 ligand-encoding vectors. Suitable host cells for the expression of glycosylated Apo-2 ligand are derived from multicellular organisms. Examples of all such host cells, including CHO cells, are described further in WO97/25428.

Host cells are transfected and preferably transformed with the above-described expression or cloning vectors for Apo-2 ligand production and cultured in nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 June 1989. In addition, plants may be transfected using ultrasound treatment as described in WO 91/00358 published 10 Jan. 1991.

For mammalian cells without such cell walls, the calcium phosphate precipitation method of Graham and van der Eb, Virology, 52:456-457 (1978) may be employed. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Prokaryotic cells used to produce Apo-2 ligand may be cultured in suitable culture media as described generally in Sambrook et al., supra. Particular forms of culture media that may be employed for culturing E. coli are described further in the Examples below. Mammalian host cells used to produce Apo-2 ligand may be cultured in a variety of culture media.

Examples of commercially available culture media include Ham's F10 (Sigma), Minimal Essential Medium (“MEM”, Sigma), RPMI-1640 (Sigma), and Dulbecco's Modified Eagle's Medium (“DMEM”, Sigma). Any such media may be supplemented as necessary with hormones and/or other growth factors (such as insulin, transferrin, or epidermal growth factor) , salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In general, principles, protocols, and practical techniques for maximizing the productivity of mammalian cell cultures can be found in Mammalian Cell Biotechnology: A Practical Approach, M. Butler, ed. (IRL Press, 1991).

In accordance with one aspect of the present invention, one or more divalent metal ions will typically be added to or included in the culture media for culturing or fermenting the host cells. The divalent metal ions are preferably present in or added to the culture media at a concentration level sufficient to enhance storage stability, enhance solubility, or assist in forming stable Apo-2L trimers coordinated by one or more zinc ions. The amount of divalent metal ions which may be added will be dependent, in part, on the host cell density in the culture or potential host cell sensitivity to such divalent metal ions. At higher host cell densities in the culture, it may be beneficial to increase the concentration of divalent metal ions. If the divalent metal ions are added during or after product expression by the host cells, it may be desirable to adjust or increase the divalent metal ion concentration as product expression by the host cells increases. It is generally believed that trace levels of divalent metal ions which may be present in typical commonly available cell culture media may not be sufficient for stable trimer formation. Thus, addition of further quantities of divalent metal ions, as described herein, is preferred.

The divalent metal ions are preferably added to the culture media at a concentration which does not adversely or negatively affect host cell growth, if the divalent metal ions are being added during the growth phase of the host cells in the culture. In shake flask cultures, it was observed that ZnSO₄ added at concentrations of greater than 1 mM can result in lower host cell density. Those skilled in the art appreciate that bacterial cells can sequester metal ions effectively by forming metal ion complexes with cellular matrices. Thus, in the cell cultures, it is preferable to add the selected divalent metal ions to the culture media after the growth phase (after the desired host cell density is achieved) or just prior to product expression by the host cells. To ensure that sufficient amounts of divalent metal ions are present, additional divalent metal ions may be added or fed to the cell culture media during the product expression phase.

The divalent metal ion concentration in the culture media should not exceed the concentration which may be detrimental or toxic to the host cells. In the methods of the invention employing the host cell, E. coli, it is preferred that the concentration of the divalent metal ion concentration in the culture media does not exceed about 1 mM (preferably, ≦1 mM). Even more preferably, the divalent metal ion concentration in the culture media is about 50 micromolar to about 250 micromolar. Most preferably, the divalent metal ion used in such methods is zinc sulfate. It is desirable to add the divalent metal ions to the cell culture in an amount wherein the metal ions and Apo-2 ligand trimer can be present at a one to one molar ratio.

The divalent metal ions can be added to the cell culture in any acceptable form. For instance, a solution of the metal ion can be made using water, and the divalent metal ion solution can then be added or fed to the culture media.

Expression of the Apo-2L may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Various labels may be employed, most commonly radioisotopes, and particularly ³²P, However, other techniques may also be employed, such as using biotin-modified nucleotides for introduction into a polynucleotide. The biotin then serves as the site for binding to avidin or antibodies, which may be labeled with a wide variety of labels, such as radionucleotides, fluorescers or enzymes. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected. Gene expression, alternatively, may be measured by immunological methods, such as immunohistochemical staining of cells or tissue sections and assay of cell culture or body fluids, to quantitate directly the expression of gene product. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like.

Antibodies useful for immunohistochemical staining and/or assay of sample fluids may be either monoclonal or polyclonal, and may be prepared in any mammal. Conveniently, the antibodies may be prepared against a native Apo-2 ligand polypeptide or against a synthetic peptide based on the DNA sequences provided herein or against exogenous sequence fused to Apo-2 ligand DNA and encoding a specific antibody epitope.

Apo-2 ligand preferably is recovered from the culture medium as a secreted polypeptide, although it also may be recovered from host cell lysates when directly produced without a secretory signal. If the Apo-2 ligand is membrane-bound, it can be released from the membrane using a suitable detergent solution (e.g. Triton-X 100) or its extracellular region may be released by enzymatic cleavage.

When Apo-2 ligand is produced in a recombinant cell other than one of human origin, the Apo-2 ligand is free of proteins or polypeptides of human origin. However, it is usually necessary to recover or purify Apo-2 ligand from recombinant cell proteins or polypeptides to obtain preparations that are substantially homogeneous as to Apo-2 ligand. As a first step, the culture medium or lysate may be centrifuged to remove particulate cell debris. Apo-2 ligand thereafter is purified from contaminant soluble proteins and polypeptides, with the following procedures being exemplary of suitable purification procedures: by fractionation on an ion-exchange column; ethanol precipitation; reverse phase HPLC; chromatography on silica or on a cation-exchange resin such as DEAE or CM; chromatofocusing; SDS-PAGE; ammonium sulfate precipitation; gel filtration using, for example, Sephadex G-75; diafiltration and protein A Sepharose columns to remove contaminants such as IgG.

In a preferred embodiment, the Apo-2 ligand can be isolated by affinity chromatography. Apo-2 ligand fragments or variants in which residues have been deleted, inserted, or substituted are recovered in the same fashion as native Apo-2 ligand, taking account of any substantial changes in properties occasioned by the variation. For example, preparation of an Apo-2 ligand fusion with another protein or polypeptide, e.g., a bacterial or viral antigen, facilitates purification; an immunoaffinity column containing antibody to the antigen can be used to adsorb the fusion polypeptide.

A protease inhibitor such as phenyl methyl sulfonyl fluoride (PMSF) also may be useful to inhibit proteolytic degradation during purification, and antibiotics may be included to prevent the growth of adventitious contaminants. One skilled in the art will appreciate that purification methods suitable for native Apo-2 ligand may require modification to account for changes in the character of Apo-2 ligand or its variants upon expression in recombinant cell culture.

During any such purification steps, it may be desirable to expose the recovered Apo-2L to a divalent metal ion-containing solution or to purification material (such as a chromatography medium or support) containing one or more divalent metal ions. In a preferred embodiment, the divalent metal ions and/or reducing agent is used during recovery or purification of the Apo-2L. Optionally, both divalent metal ions and reducing agent, such as DTT or BME, may be used during recovery or purification of the Apo-2L. It is believed that use of divalent metal ions during recovery or purification will provide for stability of Apo-2L trimer or preserve Apo-2L trimer formed during the cell culturing step.

The description below also relates to methods of producing Apo-2 ligand covalently attached (hereinafter “conjugated”) to one or more chemical groups. Chemical groups suitable for use in an Apo-2L conjugate of the present invention are preferably not significantly toxic or immunogenic. The chemical group is optionally selected to produce an Apo-2L conjugate that can be stored and used under conditions suitable for storage. A variety of exemplary chemical groups that can be conjugated to polypeptides are known in the art and include for example carbohydrates, such as those carbohydrates that occur naturally on glycoproteins, polyglutamate, and non-proteinaceous polymers, such as polyols (see, e.g., U.S. Pat. No. 6,245,901).

A polyol, for example, can be conjugated to polypeptides such as an Apo-2L at one or more amino acid residues, including lysine residues, as is disclosed in WO 93/00109, supra. The polyol employed can be any water-soluble poly(alkylene oxide) polymer and can have a linear or branched chain. Suitable polyols include those substituted at one or more hydroxyl positions with a chemical group, such as an alkyl group having between one and four carbons. Typically, the polyol is a poly(alkylene glycol), such as poly(ethylene glycol) (PEG), and thus, for ease of description, the remainder of the discussion relates to an exemplary embodiment wherein the polyol employed is PEG and the process of conjugating the polyol to a polypeptide is termed “pegylation.” However, those skilled in the art recognize that other polyols, such as, for example, poly(propylene glycol) and polyethylene-polypropylene glycol copolymers, can be employed using the techniques for conjugation described herein for PEG.

The average molecular weight of the PEG employed in the pegylation of the Apo-2L can vary, and typically may range from about 500 to about 30,000 daltons (D) . Preferably, the average molecular weight of the PEG is from about 1,000 to about 25,000 D, and more preferably from about 1,000 to about 5,000 D. In one embodiment, pegylation is carried out with PEG having an average molecular weight of about 1,000 D. Optionally, the PEG homopolymer is unsubstituted, but it may also be substituted at one end with an alkyl group. Preferably, the alkyl group is a C1-C4 alkyl group, and most preferably a methyl group. PEG preparations are commercially available, and typically, those PEG preparations suitable for use in the present invention are nonhomogeneous preparations sold according to average molecular weight. For example, commercially available PEG(5000) preparations typically contain molecules that vary slightly in molecular weight, usually ±500 D.

The Apo-2 ligand of the invention may be in various forms, such as in monomer form or trimer form (comprising three monomers). Optionally, an Apo-2L trimer will be pegylated in a manner such that a PEG molecule is linked or conjugated to one, two or each of the three monomers that make up the trimeric Apo-2L. In such an embodiment, it is preferred that the PEG employed have an average molecular weight of about 1,000 to about 5,000 D. It is also contemplated that the Apo-2L trimers may be “partially” pegylated, i.e., wherein only one or two of the three monomers that make up the trimer are linked or conjugated to PEG.

A variety of methods for pegylating proteins are known in the art. Specific methods of producing proteins conjugated to PEG include the methods described in U.S. Pat. Nos. 4,179,337, 4,935,465 and 5,849,535. Typically the protein is covalently bonded via one or more of the amino acid residues of the protein to a terminal reactive group on the polymer, depending mainly on the reaction conditions, the molecular weight of the polymer, etc. The polymer with the reactive group(s) is designated herein as activated polymer. The reactive group selectively reacts with free amino or other reactive groups on the protein. The PEG polymer can be coupled to the amino or other reactive group on the protein in either a random or a site specific manner. It will be understood, however, that the type and amount of the reactive group chosen, as well as the type of polymer employed, to obtain optimum results, will depend on the particular protein or protein variant employed to avoid having the reactive group react with too many particularly active groups on the protein. As this may not be possible to avoid completely, it is recommended that generally from about 0.1 to 1000 moles, preferably 2 to 200 moles, of activated polymer per mole of protein, depending on protein concentration, is employed. The final amount of activated polymer per mole of protein is a balance to maintain optimum activity, while at the same time optimizing, if possible, the circulatory half-life of the protein.

It is further contemplated that the Apo2L described herein may be also be linked or fused to leucine zipper sequences using techniques known in the art.

Methods for generating death receptor antibodies are also described herein. The antigen to be used for production of, or screening for, antibody may be, e.g., a soluble form of the antigen or a portion thereof, containing the desired epitope. Alternatively, or additionally, cells expressing the antigen at their cell surface can be used to generate, or screen for, antibody. Other forms of the antigen useful for generating antibody will be apparent to those skilled in the art. Preferably, the antigen is a DR4 or DR5 receptor.

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy-and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. Nos. 5,571,894; and 5,587,458. The antibody fragment may also be a “linear antibody”, e.g., as described in U.S. Pat. No. 5,641,870 for example. Such linear antibody fragments may be monospecific or bispecific.

(vi) Bispecific Antibodies

Bispecific antibodies are antibodies that have binding specificities for at least two different epitopes. Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986). According to another approach described in U.S. Pat. No. 5,731,168, the interface between a pair of antibody molecules can be engineered to maximize the percentage of heterodimers which are recovered from recombinant cell culture. The preferred interface comprises at least a part of the CH³ domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985); Shalaby et al., J. Exp. Med., 175: 217-225 (1992).

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991). Antibodies with three or more antigen binding sites are described in WO01/77342 (Miller and Presta).

The antibody used in the methods or included in the articles of manufacture herein is optionally conjugated to a cytotoxic agent.

Chemotherapeutic agents useful in the generation of such antibody-cytotoxic agent conjugates have been described above.

Conjugates of an antibody and one or more small molecule toxins, such as a calicheamicin, a maytansine (U.S. Pat. No. 5,208,020), a trichothene, and CC1065 are also contemplated herein. In one embodiment of the invention, the antibody is conjugated to one or more maytansine molecules (e.g. about 1 to about 10 maytansine molecules per antibody molecule). Maytansine may, for example, be converted to May-SS—Me which may be reduced to May-SH3 and reacted with modified antibody (Chari et al. Cancer Research 52: 127-131 (1992)) to generate a maytansinoid-antibody conjugate.

Alternatively, the antibody is conjugated to one or more calicheamicin molecules. The calicheamicin family of antibiotics is capable of producing double-stranded DNA breaks at sub-picomolar concentrations. Structural analogues of calicheamicin which may be used include, but are not limited to, γ¹¹, α₂¹, α₃¹, N-acetyl-γ₁¹, PSAG and θ^{I}₁ (Hinman et al. Cancer Research 53: 3336-3342 (1993) and Lode et al. Cancer Research 58: 2925-2928 (1998)).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP—S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates antibody conjugated with a compound with nucleolytic activity (e.g. a ribonuclease or a DNA endonuclease such as a deoxyribonuclease; DNase).

A variety of radioactive isotopes are available for the production of radioconjugated antagonists or antibodies. Examples include At²¹¹, I¹³¹, I¹²⁵, Y⁹⁰, Re¹⁸⁶ , Re188, Sm¹⁵³, Bi²¹², P³² and radioactive isotopes of Lu.

Conjugates of the antibody and cytotoxic agent may be made using a variety of bifunctional protein coupling agents such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antagonist or antibody. See WO94/11026. The linker may be a “cleavable linker” facilitating release of the cytotoxic drug in the cell. For example, an acid-labile linker, peptidase-sensitive linker, dimethyl linker or disulfide-containing linker (Chari et al. Cancer Research 52: 127-131 (1992)) may be used.

Alternatively, a fusion protein comprising the antibody and cytotoxic agent may be made, e.g. by recombinant techniques or peptide synthesis.

The antibodies of the present invention may also be conjugated with a prodrug-activating enzyme which converts a prodrug (e.g. a peptidyl chemotherapeutic agent, see WO81/01145) to an active anti-cancer drug. See, for example, WO 88/07378 and U.S. Pat. No. 4,975,278.

The enzyme component of such conjugates includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

The enzymes of this invention can be covalently bound to the antibody by techniques well known in the art such as the use of the heterobifunctional crosslinking reagents discussed above. Alternatively, fusion proteins comprising at least the antigen binding region of an antibody linked to at least a functionally active portion of an enzyme of the invention can be constructed using recombinant DNA techniques well known in the art (see, e.g., Neuberger et al., Nature, 312: 604-608 (1984)).

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol.

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Alternatively, or additionally, one may increase, or decrease, serum half-life by altering the amino acid sequence of the Fc region of an antibody to generate variants with altered FcRn binding. Antibodies with altered FcRn binding and/or serum half life are described in WO00/42072 (Presta, L.).

Formulations comprising death receptor agonists and EGFR inhibitors are also provided by the present invention. It is believed that such formulations will be particularly suitable for storage as well as for therapeutic administration. The formulations may be prepared by known techniques. For instance, the formulations may be prepared by buffer exchange on a gel filtration column.

Typically, an appropriate amount of a pharmaceutically-acceptable salt is used in the formulation to render the formulation isotonic. Examples of pharmaceutically-acceptable carriers include saline, Ringer's solution and dextrose solution. The pH of the formulation is preferably from about 6 to about 9, and more preferably from about 7 to about 7.5. It will be apparent to those persons skilled in the art that certain carriers may be more preferable depending upon, for instance, the route of administration and concentrations of death receptor agonist and EGFR inhibitor.

Therapeutic compositions can be prepared by mixing the desired molecules having the appropriate degree of purity with optional pharmaceutically acceptable carriers, excipients, or stabilizers (Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances. Carriers for topical or gel-based forms include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Formulations to be used for in vivo administration should be sterile. This is readily accomplished by filtration through sterile filtration membranes, prior to or following lyophilization and reconstitution. The formulation may be stored in lyophilized form or in solution if administered systemically. If in lyophilized form, it is typically formulated in combination with other ingredients for reconstitution with an appropriate diluent at the time for use. An example of a liquid formulation is a sterile, clear, colorless unpreserved solution filled in a single-dose vial for subcutaneous injection.

Therapeutic formulations generally are placed into a container having a sterile access port, for example, an intravenous solution bag or vial having a stopper pierceable by a hypodermic injection needle. The formulations are preferably administered as repeated intravenous (i.v.), subcutaneous (s.c.), intramuscular (i.m.) injections or infusions, or as aerosol formulations suitable for intranasal or intrapulmonary delivery (for intrapulmonary delivery see, e.g., EP 257,956).

The molecules disclosed herein can also be administered in the form of sustained-release preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot (injectable microspheres composed of lactic acid-glycolic acid copolymer and leuprolide acetate), and poly-D-(−)-3-hydroxybutyric acid (EP 133,988).

The death receptor agonists and EGFR inhibitors described herein can be employed in a variety of therapeutic applications. Among these applications are methods of treating various cancers. Diagnosis in mammals of the various pathological conditions described herein can be made by the skilled practitioner. Diagnostic techniques are available in the art which allow, e.g., for the diagnosis or detection of cancer or immune related disease in a mammal. For instance, cancers may be identified through techniques, including but not limited to, palpation, blood analysis, x-ray, NMR and the like. Immune related diseases can also be readily identified. In systemic lupus erythematosus, the central mediator of disease is the production of auto-reactive antibodies to self proteins/tissues and the subsequent generation of immune-mediated inflammation. Multiple organs and systems are affected clinically including kidney, lung, musculoskeletal system, mucocutaneous, eye, central nervous system, cardiovascular system, gastrointestinal tract, bone marrow and blood. Rheumatoid arthritis (RA) is a chronic systemic autoimmune inflammatory disease that mainly involves the synovial membrane of multiple joints with resultant injury to the articular cartilage. The pathogenesis is T lymphocyte dependent and is associated with the production of rheumatoid factors, auto-antibodies directed against self IgG, with the resultant formation of immune complexes that attain high levels in joint fluid and blood. These complexes in the joint may induce the marked infiltrate of lymphocytes and monocytes into the synovium and subsequent marked synovial changes; the joint space/fluid if infiltrated by similar cells with the addition of numerous neutrophils. Tissues affected are primarily the joints, often in symmetrical pattern. However, extra-articular disease also occurs in two major forms. One form is the development of extra-articular lesions with ongoing progressive joint disease and typical lesions of pulmonary fibrosis, vasculitis, and cutaneous ulcers. The second form of extra-articular disease is the so called Felty's syndrome which occurs late in the RA disease course, sometimes after joint disease has become quiescent, and involves the presence of neutropenia, thrombocytopenia and splenomegaly. This can be accompanied by vasculitis in multiple organs with formations of infarcts, skin ulcers and gangrene. Patients often also develop rheumatoid nodules in the subcutis tissue overlying affected joints; the nodules late stage have necrotic centers surrounded by a mixed inflammatory cell infiltrate. Other manifestations which can occur in RA include: pericarditis, pleuritis, coronary arteritis, interstitial pneumonitis with pulmonary fibrosis, keratoconjunctivitis sicca, and rheumatoid nodules.

The death receptor agonists and EGFR inhibitors can be administered in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Optionally, administration may be performed through mini-pump infusion using various commercially available devices.

Effective dosages and schedules for administering the death receptor agonist may be determined empirically, and making such determinations is within the skill in the art. Single or multiple dosages may be employed. It is presently believed that an effective dosage or amount of the death receptor agonist used alone may range from about 1 μg/kg to about 100 mg/kg of body weight or more per day. Interspecies scaling of dosages can be performed in a manner known in the art, eg, as disclosed in Mordenti et al., Pharmaceut. Res., 8:1351 (1991).

When in vivo administration of the death receptor agonist is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue. Those skilled in the art will understand that the dosage of the death receptor agonist that must be administered will vary depending on, for example, the mammal which will receive the death receptor agonist, the route of administration, and other drugs or therapies being administered to the mammal.

In the methods of the invention, when the EGFR inhibitor is recombinant humanized monoclonal antibody anti-EGFR, e.g. C225 (cetuximab) or ABX-EGF, the course of therapy generally employed is from about 1 to about 1000 mg/M²; in particular about 60 to about 600 mg/M²; in particular about 150 to about 500 mg/M² of body surface area. In a particular embodiment, the course therapy employed consists of a loading dose of about 400 mg/M², followed by weekly maintenance dosage of about 180-250 mg/M². According to particular embodiments, patients are given an injection of the antibody as a weekly, dose escalating 4-week protocol, with doses up to 200 mg/M². If the disease is stabilized, then a further 8-week course can begin. In the methods of the subject invention, for the administration of small molecule EGFR inhibitors e.g. OSI-774 and ZD-1839 the course of therapy generally employed is from about 1 to about 1000 mg/day. In a particular embodiment the small molecule EGFR inhibitor is administered in an amount of about 10 to about 750 mg/day; about 50 to about 500 mg/day; or about 100 to 250 mg/day.

It is contemplated that yet additional therapies may be employed in the methods. The one or more other therapies may include but are not limited to, administration of radiation therapy, cytokine(s), growth inhibitory agent(s), chemotherapeutic agent(s), cytotoxic agent(s), tyrosine kinase inhibitors, ras farnesyl transferase inhibitors, angiogenesis inhibitors, and cyclin-dependent kinase inhibitors which are known in the art and defined further with particularity in Section I above.

Preparation and dosing schedules for chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of the Apo2L variant, or may be given simultaneously therewith.

Sometimes, it may be beneficial to also administer one or more cytokines or growth inhibitory agent.

The death receptor agonists and EGFR inhibitors (and one or more other therapies) may be administered concurrently (simultaneously) or sequentially. In particular embodiments, Apo2L/TRAIL and an EGFR inhibitor are administered concurrently. In another embodiment, Apo2L/TRAIL is administered prior to administration of an EGFR inhibitor. In another embodiment, an EGFR inhibitor is administered prior to Apo2L/TRAIL. Following administration, treated cells in vitro can be analyzed. Where there has been in vivo treatment, a treated mammal can be monitored in various ways well known to the skilled practitioner. For instance, tumor cells can be examined pathologically to assay for necrosis or serum can be analyzed for immune system responses.

An article of manufacture such as a kit containing death receptor agonists and EGFR inhibitors useful in the treatment of the disorders described herein comprises at least a container and a label. Suitable containers include, for example, bottles, vials, syringes, and test tubes. The containers may be formed from a variety of materials such as glass or plastic. The label on, or associated with, the container indicates that the formulation is used for treating the condition of choice. The article of manufacture may further comprise a container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use. The article of manufacture may also comprise a container with another active agent as described above.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Preparation of Compound of Formula 4

Reaction:

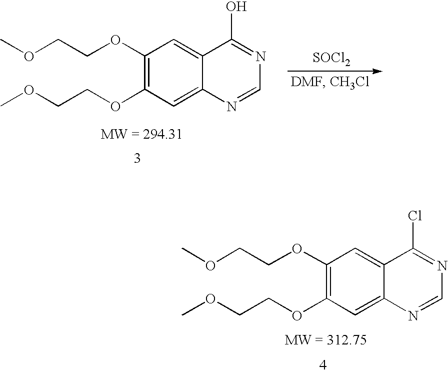

The following materials were used in the synthesis of the compound of formula 4:

| Materials | Quantity | Units | Equivalents/volumes | |
|---|---|---|---|---|
| Compound of formula 3 | 88.0 | kg | 1 | equivalent |
| Thionyl chloride | 89.0 | kg | 2.5 | equivalents |
| Dimethylformamide | 11 | kg | 0.5 | equivalent |
| methylene chloride | 880.0 | L | 10.5 | L/kg |
| 50% NaOH soln | as required | L | 1 | equivalent |
| Heptane | 880.0 | L | 10 | L/kg |

The following procedure is exemplary of the procedure to follow in the synthesis of the formula 4 compound:

88.0 kg of the compound of formula 3, 880.0 L methylene chloride, and 11.0 kg of dimethylformamide were charged to a clean, dry, glass-lined vessel under nitrogen atmosphere. 89 Kg of thionyl chloride were added to the mix while it is maintained at a temperature of a less than 30° C. during the charge. The contents of the reaction vessel were then heated for a minimum of five hours at reflux temperature before sampling for reaction completion and the pH is adjusted to be maintained between 7.0 to 8.0, by using 50% NaOH, as required and the temperature of the reaction mixture is maintained at less than 25° C. The biphasic mixture is stirred for fifteen to twenty minutes and allowed to settle for a minimum of thirty minutes. The layers were separated and the organic layer was concentrated to ⅓ of its volume by removing methylene chloride. 880 L heptane was added with continued distillation of the remaining methylene chloride until the distillate reaches a temperature between 65 and 68° C. The mixture was then cooled to between 10 to 15° C. over hours and granulated for a minimum of 1 hour with the solids being isolated by filtration and washed with 220 L heptane. The solids (formula 4 compound) were dried in a vacuum drier at 45 to 50° C.

### Example 2

### Alternative Preparation of Compound of Formula 4

In the reaction shown in Example 1, sodium bicarbonate may successfully be used instead of sodium hydroxide as shown in this Example.

| Materials | Quantity | Units | Equivalents/Volumes | |
|---|---|---|---|---|
| Compd 3 | 30.0 | kg | 1 | equivalent |
| Thionyl chloride | 36.4 | kg | 3 | equivalents |
| Dimethylformamide | 3.75 | kg | 0.5 | equivalent |
| methylene chloride | 300 | L | 10 | L/kg |
| 50% NaOH soln | as required | L | | |
| Heptane | 375 | L | 12.5 | L/kg |
| Heptane (wash) | 90 | L | 3 | L/kg |
| Sodium Bicarbonate | 64.2 | Kg | 7.5 | equivalents |

30.0 kg of the compound of formula 3, 300.0 L methylene chloride, and 3.75 kg of dimethylformamide were charged to a clean, dry, glass-lined vessel under a nitrogen atmosphere.

36.4 kg of thionyl chloride was added to the mix while it was maintained at a temperature of less than 30° C. during the charge. The contents of the reaction vessel were then heated at reflux temperature for 13h before sampling for reaction completion. The reaction mixture was cooled to 20-25° C. and added slowly to a stirred solution of sodium bicarbonate 64.2 kg and water 274L cooled to 4° C. so that the temperature was maintained at less than 10° C. The final pH of the mixture was adjusted to within the range 7.0 to 8.0 by using 50% sodium hydroxide solution as required. The biphasic mixture was stirred for fifteen to twenty minutes and allowed to settle for a minimum of thirty minutes at 10-20° C. The layers were separated and the organic layer was concentrated to ⅓ of its volume by removing methylene chloride. 375L of heptane was added with continued distillation of the remaining methylene chloride until the distillate reached a temperature between 65 and 68° C. The mixture was then cooled to 0 to 5° C. over 4 hours and granulated for a minimum of 1 hour with the solids being isolated by filtration and washed with 90L heptane. The solids (formula 4 compound) were dried in a vacuum drier to 50° C.

### Example 3

### Preparation of Compounds 6 and 2 (Step 2)

Reaction:

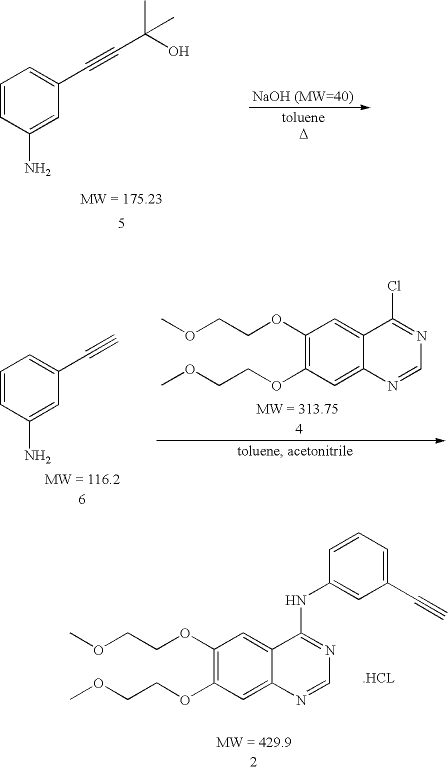

The following materials were used in the synthesis of the compound of formula 6, as intermediate, and the compound of formula 2:

| Materials | Quantity | Units | Equivalents/Volumes | |
|---|---|---|---|---|
| Comp 5 | 61.1 | kg | 1.2 | equivalents |
| Toluene | 489 | L | 8 | L/kg (WRT to comp 5) |
| NaOH pellets | 4.5 | kg | 0.16 | equivalents |
| Filteraid | 0.5 | kg | 0.017 | kg/kg (WRT to comp 5) |
| Comp 4 | 90.8 | kg | 1.0 | equivalent |
| Acetonitrile | 732 | L | 12 | L/kg (WRT to compd 5) |

The following procedure is exemplary of the procedure to follow in the synthesis of the formula 2 compound and intermediate compound of formula 6:

61.1 kg of formula 5 compound, 4.5 kg sodium hydroxide pellets and 489 L toluene were charged to a clean, dry, reaction vessel under nitrogen atmosphere and the reaction temperature is adjusted to between 105 to 108° C. Acetone was removed over four hours by atmospheric distillation while toluene is added to maintain a minimum volume of 6 L of solvent per kg of formula 5 compound. The reaction mixture was then heated at reflux temperature, returning distillates to pot, until the reaction was complete. The mixture was then cooled to between 20 to 25° C., at which time a slurry of 40.0 L toluene and 0.5 kg filteraid was charged to the reaction mixture and the mixture was agitated for ten to fifteen minutes. The resultant material was filtered to remove filteraid, and the cake is washed with 30 L toluene (compound of formula 6).

The filtrate (compound of formula 6) was placed in a clean, dry reaction vessel under nitrogen atmosphere, and 90.8 kg of the compound of formula 4 was charged into the reaction vessel together with 732 L acetonitrile. The reaction vessel was heated to reflux temperature and well agitated. Agitator speed was lowered when heavy solids appear. When the reaction was complete, the contents of reaction vessel were cooled to between to 25° C. over three to four hours and the contents were agitated for at least one hour at a temperature between 20 and 25° C. The solids (compound of formula 2, polymorph A form, or mixture of polymorph A and B) were then isolated by filtration and the filter cake was washed with two portions of 50 L acetonitrile and dried under vacuum at a temperature between 40 and 45° C.

It has been discovered that the production of the A polymorph is favored by the reduction of the amount of acetonitrile relative to toluene, and particularly favored if isopropanol is used in place of acetonitrile. However, the use of isopropanol or other alcohols as cosolvents is disfavored because of the propensity to form an ether linkage between the alcoholic oxygen and the 4-carbon of the quinazoline, instead of the desired ethynyl phenyl amino moiety.

It has been further discovered that adjusting the pH of the reaction to between pH 1 and pH 7, particularly between pH 2 and pH 5, for example, between pH 2.5 and pH 4, such as pH 3, will improve the rate of the reaction.

### Example 5

### Recrystallization of Compound of Formula 2 (Which May be in Polymorph A Form or a Mixture of Polymorphs A and B) to Polymorph B (Step 3)

Reaction:

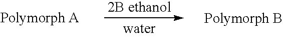

The following materials were used in the conversion of polymorph A (or mixtures of polymorphs A and B) to polymorph B of the compound of formula 2:

| Materials | Quantity | Units | EquivalentsNolumes | |
|---|---|---|---|---|
| Polymorph A (comp 2) | 117.6 | kg | 1 | equivalent |
| 2B-ethanol | 1881.6 | L | 16 | L/kg |
| Water | 470.4 | L | 4 | L/kg |

The following procedure is exemplary of procedures used to convert polymorph A (or mixtures of polymorphs A and B) into the more thermodynamically stable polymorph B of the compound of formula 2:

117.6 kg of the polymorph A (or mixtures of polymorphs A and B) were charged to a clean, dry, reaction vessel together 1881.6 L 2B-ethanol and 470.4 L water under a nitrogen atmosphere. The temperature was adjusted to reflux (−80° C.) and the mixture was agitated until the solids dissolve. The solution was cooled to between 65 and 70° C. and clarified by filtration. With low speed agitation, the solution was further cooled to between 50 and 60° C. over a minimum time of 2 hours and the precipitate was granulated for 2 hours at this temperature. The mixture was further cooled to between 0 and 5° C. over a minimum time of 4 hours and granulated for a minimum of 2 hours at this temperature. The solids (polymorph B) were isolated by filtration and washed with at least 100 L 2B-ethanol. The solids were determined to be crystalline polymorph B form of [6,7-bis(2-methoxyethoxy)quinazolin-4-yl]-(3-ethynyiphenyl)-amine hydrochloride substantially free of the polymorph A form. The solids obtained by this method are substantially homogeneous polymorph B form crystals relative to the polymorph A form. The method allows for production of polymorph B in an amount at least 70% by weight, at least 80% by weight, at least 90% by weight, at least 95% by weight, and at least 98% by weight relative to the weight of the polymorph A. It is to be understood that the methods described herein are only exemplary and are not intended to exclude variations in the above parameters which allow the production of polymorph B in varying granulations and yields, according to the desired storage, handling and manufacturing applications of the compound. The solids were vacuum dried at a temperature below 50° C. and the resultant product was milled to provide the polymorph B in usable form.

Polymorph B exhibits an X-ray powder diffraction pattern having characteristic peaks expressed in degrees 2-theta at approximately 6.26, 12.48, 13.39, 16.96, 20.20, 21.10, 22.98, 24.46, 25.14 and 26.91.

### Example 5

### Analysis of Apo2L/TRAIL Receptor and EGFR Expression in H460 Cell Lines

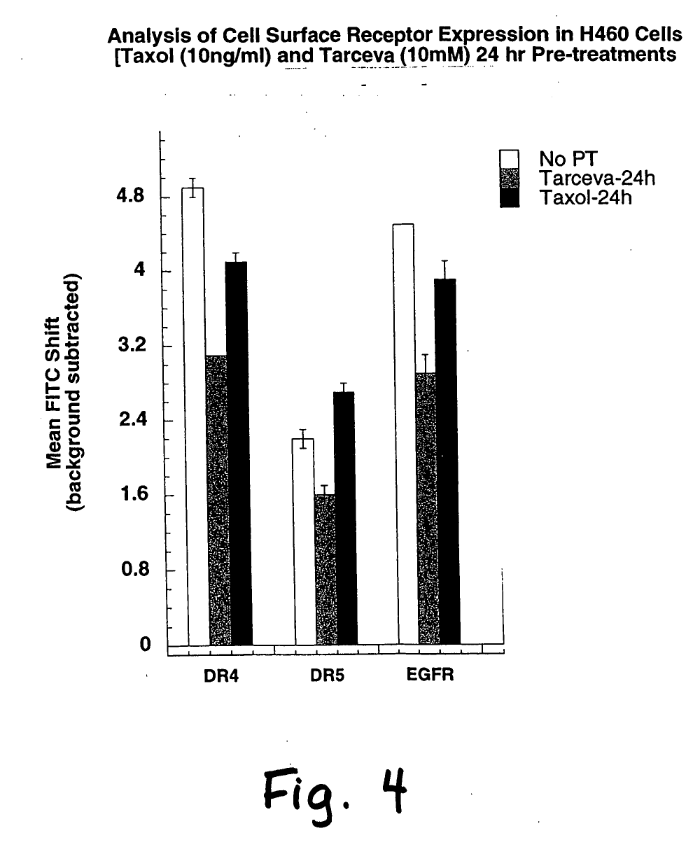

To examine the cell-surface expression of Apo2L/TRAIL receptors (DR4, DR5) and EGFR in human non-small lung cancer cell lines, the H460 cell line (ATCC) was analyzed by FACS using monoclonal antibodies specific for DR4 (mAb 4H6.17.8; ATCC HB-12455), DR5 (mAb 3H3.14.5; HB-12534), and EGFR. The cells were either pretreated with Tarceva™, Taxol®, or alternatively received no pretreatment, as indicated in FIG. 4. The bar diagram in FIG. 4 illustrates the respective receptor expression levels.

### Example 6

### Effects of Apo2L/TRAIL, Tarceva™, or Combination Treatment on the Growth of Cancer Cells in Vitro

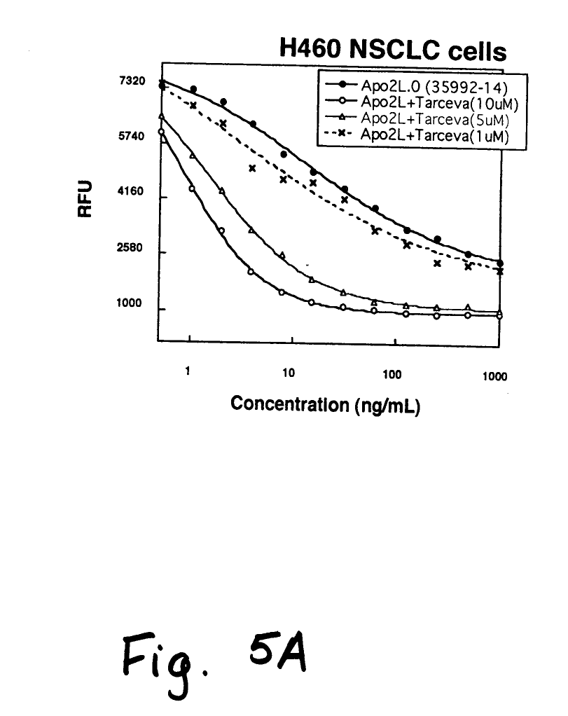

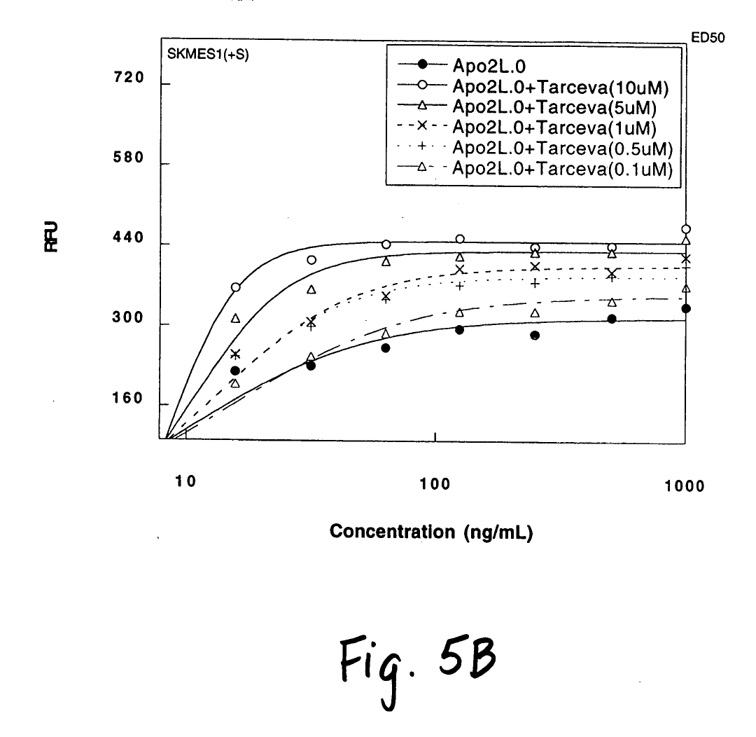

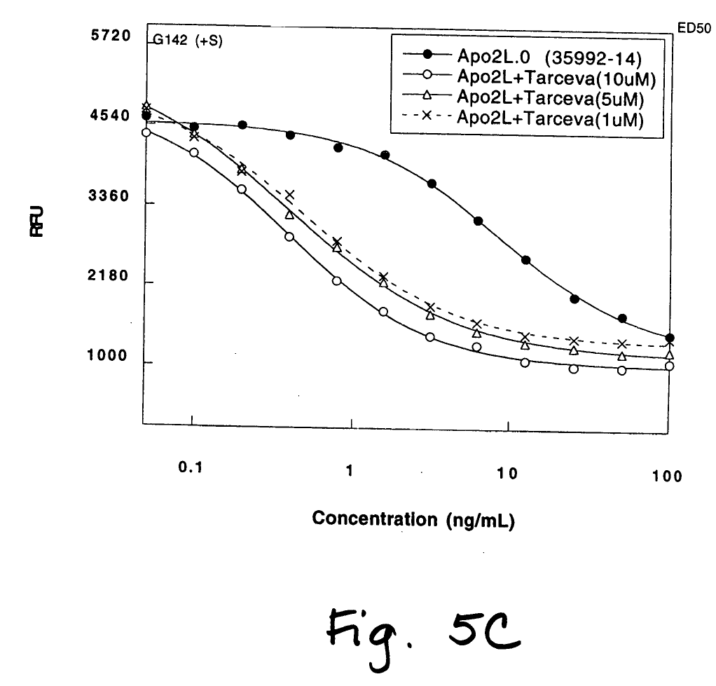

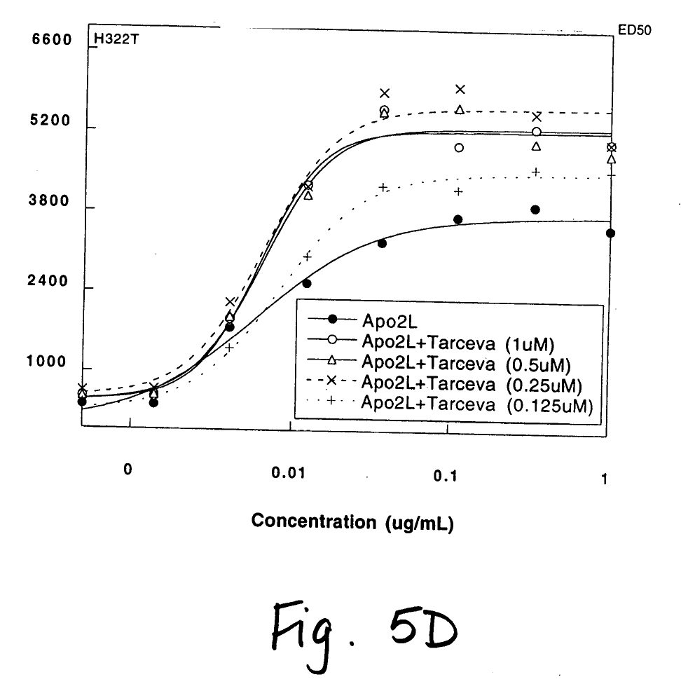

Serial dilutions of H460, SKMES1, G142, and H332T cells were performed in 96-well tissue culture plates (Falcon). The effects of Apo-2 ligand (amino acids 114-281, described in PCT US00/17579) and Tarceva™ were tested, as described in FIGS. 5A-5D. The plates were incubated at 37° C. for 24 hours. AlamarBlue (Trek Diagnostic Systems, Inc.) was added to the wells for the last 3 hours of the 24 hours incubation time. Fluorescence was read using a 96-well fluorometer with excitation at 530 nm and emission of 590 nm. The results are expressed in relative fluorescence units (RFU). For data analysis the 4-parameter curve fitting program (Kaleidagraph) was used.

The results of the bioassays are shown in FIGS. 5A-5D.

### Example 7

### Effect of Apo2L/TRAIL, Tarceva™, or Combination Treatment on the Growth of H460 Tumor Xenografts in Vivo

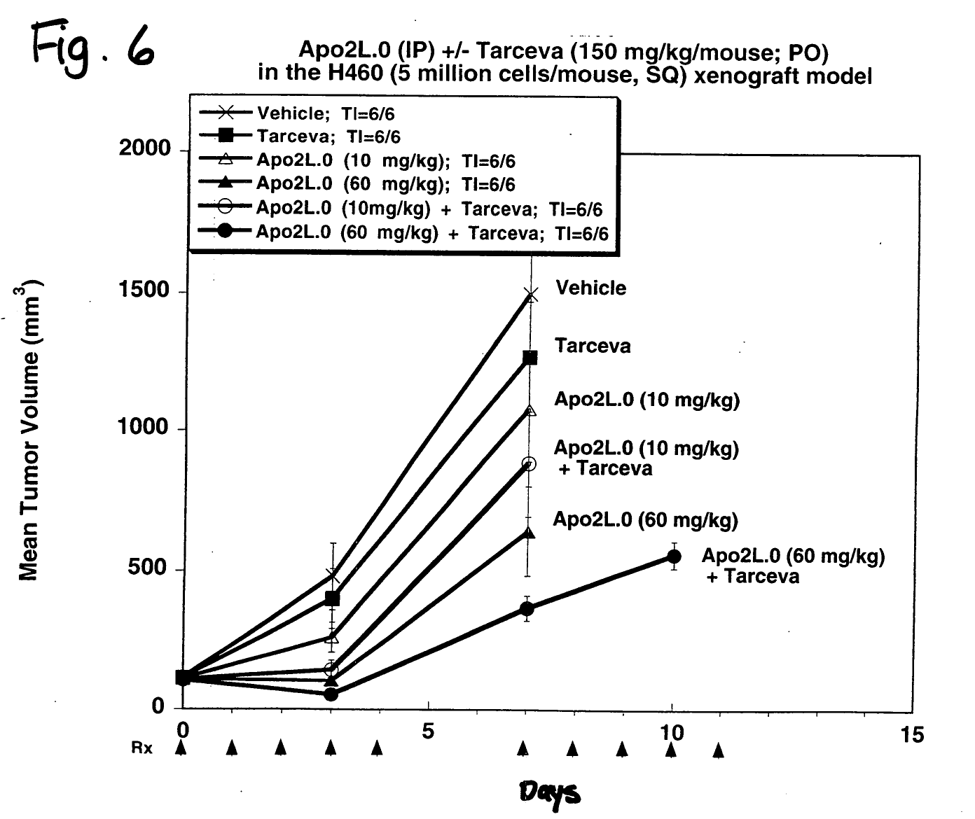

Mice were injected subcutaneously with H460 non-small lung cancer cells (ATCC) (5 million cells per mouse) . The mice were then divided into 6 study groups (6 mice per group) and treated with vehicle, Tarceva™ (Genentech, Inc.), Apo2L.0 (Apo2L/TRAIL polypeptide consisting of amino acids 114-281 of FIG. 1 (see Ashkenazi et al., J. Clin. Invest., 104:155-162 (1999)) or combinations thereof, as provided in FIG. 6. Tumors in vehicle-treated mice grew rapidly, while Apo2L/TRAIL and Tarceva™ treatment markedly delayed tumor growth.

What is claimed is:

1. A method of enhancing apoptosis in one or more mammalian cells, comprising exposing said cells to an effective amount of death receptor agonist and EGFR inhibitor.

2. The method of claim 1, wherein said cells are exposed sequentially to the death receptor agonist and the EGFR inhibitor.

3. The method of claim 1, wherein said cells are exposed to the EGFR inhibitor prior to being exposed to the death receptor agonist.

4. The method of claim 1, wherein said death receptor agonist comprises Apo2L/TRAIL polypeptide.

5. The method of claim 1, wherein said cells are exposed simultaneously to the EGFR inhibitor and the death receptor agonist.

6. The method of claim 1, wherein said death receptor agonist is a DR4 agonist antibody or DR5 agonist antibody.

7. The method of claim 1, wherein said EGFR inhibitor has the general formula I 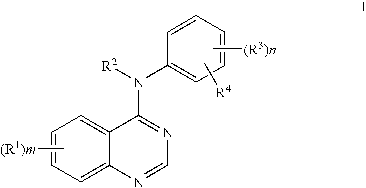
wherein:
X is halo or hydroxy;
m is 1, 2, or 3;
each R¹ is independently selected from the group consisting of hydrogen, halo, hydroxy, hydroxyamino, carboxy, nitro, guanidino, ureido, cyano, trifluoromethyl, and —(C₁ -C₄ alkylene)-W-(phenyl) wherein W is a single bond, O, S or NH;
or each R¹ is independently selected from R⁹ and C₁-C₄ alkyl substituted by cyano, wherein R⁹ is selected from the group consisting of R⁵, —OR⁶, —NR⁶ R⁶, —C(O)R⁷, —NHOR⁵, —OC(O)R⁶, cyano, A and —YR⁵; R⁵ is C₁-C₄ alkyl; R⁶ is independently hydrogen or R⁵; R⁷ is R⁵, —OR⁶ or —NR⁶R⁶; A is selected from piperidino, morpholino, pyrrolidino, 4-R⁶ -piperazin-1-yl, imidazol-1-yl, 4-pyridon-1-yl, —(C₁ -C₄ alkylene) (CO2H), phenoxy, phenyl, phenylsulfanyl, C₂-C₄ alkenyl, and —(C₁-C₄ alkylene)C(O)NR⁶R⁶; and Y is S, SO, or SO₂; wherein the alkyl moieties in R⁵, —OR⁶ and —NR⁶R⁶ are optionally substituted by one to three halo substituents and the alkyl moieties in R⁵, —OR⁶ and —NR⁶R⁶ are optionally substituted by 1 or 2 R⁹ groups, and wherein the alkyl moieties of said optional substituents are optionally substituted by halo or R⁹, with the proviso that two heteroatoms are not attached to the same carbon atom;
or each R¹ is independently selected from —NHSO₂R⁵, phthalimido-(C₁-C₄) -alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄) -alkanoylamino wherein R¹⁰ is selected from halo, —OR⁶, C₂-C₄ alkanoyloxy, —C(O)R⁷, and —NR⁶R⁶; and wherein said —NHSO₂R⁵, phthalimido-(C₁-C₄-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino R¹ groups are optionally substituted by 1 or 2 substituents independently selected from halo, C₁-C₄ alkyl, cyano, methanesulfonyl and C₁-C₄ alkoxy;
or two R¹ groups are taken together with the carbons to which they are attached to form a 5-8 membered ring that includes 1 or 2 heteroatoms selected from O, S and N;
R² is hydrogen or C₁-C₆ alkyl optionally substituted by 1 to 3 substituents independently selected from halo, C₁-C₄ alkoxy, —NR⁶R⁶, and —SO₂R¹;
n is 1 or 2 and each R³ is independently selected from hydrogen, halo, hydroxy, C₁-C₆ alkyl, —NR⁶R⁶, and C₁-C₄ alkoxy, wherein the alkyl moieties of said R³ groups are optionally substituted by 1 to 3 substituents independently selected from halo, C₁-C₄ alkoxy, —NR⁶R⁶, and —SO₂R; and,
R⁴ is azido or -(ethynyl)-R¹¹ wherein R¹¹ is hydrogen or C₁-C₆ alkyl optionally substituted by hydroxy, —OR⁶, or —NR⁶R⁶.

8. The method of claim 7, wherein said EGFR inhibitor is N-(3-ethynylphenyl)-6,7-bis(2-methoxyethoxy)-4-quinazolinamine.

9. The method of claim 7, wherein said EGFR inhibitor is Tarceva™.

10. The method of claim 1, wherein said Apo2L/TRAIL is a fragment of the polypeptide of SEQ ID NO:1.

11. The method of claim 10, wherein said Apo2L/TRAIL fragment comprises the extracellular domain of the polypeptide of SEQ ID NO:1.

12. The method of claim 1, wherein said death receptor agonist is an Apo2L/TRAIL polypeptide variant having at least about 90% amino acid sequence identity with the extracellular domain of SEQ ID NO:1.

13. The method of claim 10, wherein said fragment comprises amino acids 114-281 of SEQ ID NO:1.

14. The method of claim 12, wherein said Apo2L/TRAIL variant has at least about 95% amino acid sequence identity with the extracellular domain of SEQ ID NO:1.

15. The method of claim 13, wherein said Apo2L/TRAIL fragment comprising amino acids 114-281 of SEQ ID NO:1 is linked to one or more polyethylene glycol (PEG) molecules.

16. A method of treating a proliferative disorder in a mammal comprising administering to said mammal Apo2L/TRAIL and an EGFR inhibitor.

17. The method of claim 16, wherein said Apo2L/TRAIL and an EGFR inhibitor are administered simultaneously.

18. The method of claim 16, wherein said Apo2L/TRAIL is administered prior to said EGFR inhibitor

19. The method of claim 16, wherein said EGFR inhibitor is administered prior to said Apo2L/TRAIL.

20. The method of claim 1 wherein said proliferative disorder is cancer.

21. The method of claim 20, wherein said cancer is selected from the group consisting of small-cell lung cancer, non-small cell lung cancer, colon cancer, colorectal cancer, and pancreatic cancer.

22. The method of claim 21, wherein said cancer is colon cancer, colorectal cancer, small-cell lung cancer or non-small cell lung cancer.

23. The method of claim 16, wherein said EGFR inhibitor is a compound of the general formula I: 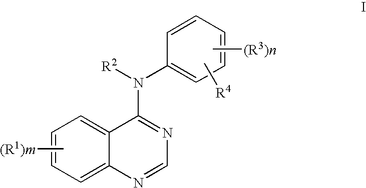
wherein:
X is halo or hydroxy;
m is 1, 2, or 3;
each R¹ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH;
or each R¹ is independently selected from R⁹ and C₁-C₄ alkyl substituted by cyano, wherein R⁹ is selected from the group consisting of R⁵, —OR⁶, —NR⁶R⁶, —C(O)R⁷, —NHOR⁵, —OC(O)R⁶, cyano, A and —YR⁵; R⁵ is C₁-C₄ alkyl; R⁶ is independently hydrogen or R⁵; R⁷ is R⁵, —OR⁶ or —NR⁶R⁶; A is selected from piperidino, morpholino, pyrrolidino, 4-R⁶-piperazin-1-yl, imidazol-1-yl, 4-pyridon-1-yl, —(C₁-C₄ alkylene) (CO2H), phenoxy, phenyl, phenylsulfanyl, C₂-C₄ alkenyl, and —(C₁-C₄ alkylene)C(O)NR⁶R⁶; and Y is S, SO, or SO₂; wherein the alkyl moieties in R⁵, —OR⁶ and —NR⁶R⁶ are optionally substituted by one to three halo substituents and the alkyl moieties in R⁵, —OR⁶ and —NR⁶R⁶ are optionally substituted by 1 or 2 R⁹ groups, and wherein the alkyl moieties of said optional substituents are optionally substituted by halo or R⁹, with the proviso that two heteroatoms are not attached to the same carbon atom;
or each R¹ is independently selected from —NHSO₂R⁵, phthalimido-(C₁-C₄) -alkylsulfonylamino, benzamido, benzenesultonylamino, 3-phenylureido, 2-oxopyrrolidin-1-yl, 2,5-dioxopyrrolidin-1-yl, and R¹⁰—(C₂-C₄)-alkanoylamino wherein R¹⁰ is selected from halo, —OR⁶, C₂-C₄ alkanoyloxy, —C(O)R⁷, and —NR⁶R⁶; and wherein said —NHSO₂R⁵, phthalimido-(C₁-C₄-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino R¹ groups are optionally substituted by 1 or 2 substituents independently selected from halo, C₁-C₄ alkyl, cyano, methanesulfonyl and C₁-C₄ alkoxy;
or two R¹ groups are taken together with the carbons to which they are attached to form a 5-8 membered ring that includes 1 or 2 heteroatoms selected from O, S and N;
R² is hydrogen or C₁-C₆ alkyl optionally substituted by 1 to 3 substituents independently selected from halo, C₁-C₄ alkoxy, —NR⁶R⁶, and —SO₂R⁵;
n is 1 or 2 and each R³ is independently selected from hydrogen, halo, hydroxy, C₁-C₆ alkyl, —NR⁶R⁶, and C₁-C₄ alkoxy, wherein the alkyl moieties of said R³ groups are optionally substituted by 1 to 3 substituents independently selected from halo, C₁-C₄ alkoxy, —NR⁶R⁶, and —SO₂R; and,
R⁴ is azido or -(ethynyl)-R¹¹ wherein R¹¹ is hydrogen or C₁-C₆ alkyl optionally substituted by hydroxy, —OR⁶, or —NR⁶R⁶.

24. The method of claim 16, wherein said EGFR inhibitor is N-(3-ethynylphenyl)-6,7-bis(2-methoxyethoxy)-4-quinazolinamine.

25. The method of claim 16, wherein said EGFR inhibitor is Tarceva™.

26. A method of treating cancer cells, comprising exposing mammalian cancer cells to a synergistic effective amount of death receptor agonist and EGFR inhibitor.

27. The method of claim 26 wherein said death receptor agonist is an anti-DR5 or anti-DR4 receptor monoclonal antibody.

28. The method of claim 26 wherein said death receptor agonist is Apo-2/TRAIL polypeptide.

29. The method of claim 26 wherein said cancer cells are exposed to said synergistic effective amount of death receptor agonist and EGFR inhibitor in vivo.

30. The method of claim 27 wherein said death receptor antibody is a chimeric antibody or a humanized antibody.

31. The method of claim 27 death receptor antibody is a human antibody.

32. The method of claim 26 wherein said death receptor agonist is an antibody which cross-reacts with more than one Apo-2 ligand receptor.

33. The method of claim 26 wherein said cancer cells are colon cancer cells, colorectal cancer cells, small-cell lung cancer cells or non-small cell lung cancer cells.

34. The method of claim 26 further comprising exposing the cancer cells to one or more growth inhibitory agents.

35. The method of claim 26 further comprising exposing the cells to radiation.

36. The method of claim 27 wherein said DR5 antibody has a DR5 receptor binding affinity of 10⁸ M⁻¹ to 10¹² M⁻¹.

37. The method of claim 26 wherein said death receptor agonist is expressed in a recombinant host cell selected from the group consisting of a CHO cell, yeast cell and E. coli.

38. The method of claim 26 wherein said EGFR inhibitor is Tarceva™.


SEQUENCE LISTING (6 sequences)

SEQ ID NO: 1 - protein, 281 aa, Homo sapiens [sequence omitted]

SEQ ID NO: 2 - DNA, 1042 bp, Homo sapiens
  Unsure 447: Unknown base
tttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtccncagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaa

SEQ ID NO: 3 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSERPGACNRCTEGVGYTNASNNLFACLPCTACKSDEEERSPCTTTRNTACQCKPGTFRNDNSAEMCRKCSTGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAKEKIQDLLVDSGKFIYLEDGTGSAVSLE

SEQ ID NO: 4 - DNA, 1407 bp, Homo sapiens
atggcgccaccaccagctagagtacatctaggtgcgttcctggcagtgactccgaatcccgggagcgcagcgagtgggacagaggcagccgcggccacacccagcaaagtgtggggctcttccgcggggaggattgaaccacgaggcgggggccgaggagcgctccctacctccatgggacagcacggacccagtgcccgggcccgggcagggcgcgccccaggacccaggccggcgcgggaagccagccctcggctccgggtccacaagaccttcaagtttgtcgtcgtcggggtcctgctgcaggtcgtacctagctcagctgcaaccatgatcaatcaattggcacaaattggcacacagcaatgggaacatagccctttgggagagttgtgtccaccaggatctcatagatcagaacgtcctggagcctgtaaccggtgcacagagggtgtgggttacaccaatgcttccaacaatttgtttgcttgcctcccatgtacagcttgtaaatcagatgaagaagagagaagtccctgcaccacgaccaggaacacagcatgtcagtgcaaaccaggaactttccggaatgacaattctgctgagatgtgccggaagtgcagcacagggtgccccagagggatggtcaaggtcaaggattgtacgccctggagtgacatcgagtgtgtccacaaagaatcaggcaatggacataatatatgggtgattttggttgtgactttggttgttccgttgctgttggtggctgtgctgattgtctgttgttgcatcggctcaggttgtggaggggaccccaagtgcatggacagggtgtgtttctggcgcttgggtctcctacgagggcctggggctgaggacaatgctcacaacgagattctgagcaacgcagactcgctgtccactttcgtctctgagcagcaaatggaaagccaggagccggcagatttgacaggtgtcactgtacagtccccaggggaggcacagtgtctgctgggaccggcagaagctgaagggtctcagaggaggaggctgctggttccagcaaatggtgctgaccccactgagactctgatgctgttctttgacaagtttgcaaacatcgtgccctttgactcctgggaccagctcatgaggcagctggacctcacgaaaaatgagatcgatgtggtcagagctggtacagcaggcccaggggatgccttgtatgcaatgctgatgaaatgggtcaacaaaactggacggaacgcctcgatccacaccctgctggatgccttggagaggatggaagagagacatgcaaaagagaagattcaggacctcttggtggactctggaaagttcatctacttagaagatggcacaggctctgccgtgtccttggagtga

SEQ ID NO: 5 - protein, 411 aa, Homo sapiens [sequence omitted]

SEQ ID NO: 6 - protein, 440 aa, Homo sapiens [sequence omitted]